(19) United States
(12) Patent Application Publication
(10) Pub. No.: US 2025/0205117 A1
(43) Pub. Date: Jun. 26, 2025

(54) NIPPLE SHIELD, SYSTEM AND METHOD TO DETECT A MILK FLOW RATE FROM A MOTHER'S BREAST

(71) Applicant: KAIZEN BIO-TECH (2011) LTD., Caesarea (IL)

(72) Inventors: Haim DAHAN, Raanana (IL); Adir Kan, Natanya (IL); Oded Hadomi, Even yehuda (IL); Abi Zakay, Tel Aviv (IL); Mario Meschiani, Tel Aviv (IL); Ali Sagas, Kafr Kama (IL)

(21) Appl. No.: 19/033,364

(22) Filed: Jan. 21, 2025

Related U.S. Application Data
(63) Continuation-in-part of application No. 29/959,730, filed on Aug. 27, 2024, now Pat. No. D058823.
(63) Continuation-in-part of application No. 18/277,383, filed on Aug. 16, 2023.
(60) Provisional application No. 63/223,616, filed on Jul. 20, 2021.

(51) Int. Cl. A61J 13/00; A61L 2/14

(52) U.S. Cl. CPC A61J 13/00; A61J 2200/70; A61L 2/14; A61L 2202/24

(57) ABSTRACT

A nipple shield adapted to provide a visual indication of a milk flow from a breast to an infant during nursing. The nipple shield includes a wide base having a protrusion disposed in a central region of the wide base, and a single channel passageway disposed within the nipple shield. The wide base is adapted to lie securely against a nursing mother's breast. The single channel passageway fluidly connects an inlet opening in a lower surface of the protrusion to an outlet opening on an upper surface of the protrusion. The single channel passageway extends from the inlet opening to a remote location so that all of the milk flow from the inlet opening in the single channel passageway can be visually observed when a mouth of the infant covers a portion of the protrusion.

### CROSS REFERENCE TO RELATED APPLICATIONS

This application incorporates and claims the benefit of the filing date of U.S. Design patent application Ser. No. 29/959,730, entitled “NIPPLE SHIELD” filed Aug. 27, 2024, now U.S. Design Patent D1058823, and U.S. patent application Ser. No. 18/277,383, entitled “DEVICE FOR FLOW DETECTION OF MOTHER'S MILK” filed Feb. 16, 2022, which claims priority to U.S. Provisional Application Ser. No. 63/223,616, entitled “DEVICE FOR FLOW DETECTION OF MOTHER'S MILK” filed Jul. 20, 2021, the entirety of which is incorporated herein by reference.

### TECHNICAL FIELD

The present disclosure describes technology related to the field of detection of a flow of milk from a nursing mother, and in particular to a nipple shield and related detection units that analyzes and visually depicts the indication and presence of a milk flow from a mother to a nursing infant.

### BACKGROUND

The desire of a nursing mother to receive an indication of whether a baby is actually intaking milk during a breastfeeding session is of particular interest to the nursing mother, especially in the first few weeks of an infant's life. Babies may be inclined to suck on a breast as a soothing mechanism, giving the mother the false impression that the baby is intaking milk, when in fact, they are not. Another concern is that some mothers do not have an adequate milk supply, especially before breast-feeding has been well-established, and consequently a hungry infant may suckle on the breast for long periods of time, without receiving an adequate supply of milk. Additionally, some babies appear to fall asleep during nursing, and the mother may not realize that they are still feeding.

In order to give an indication of whether milk is being supplied to a baby, various complex measurement systems have been previously proposed. One technique employed is weight subtraction before and after feeding, which is bothersome, time consuming and inaccurate. In this technique, the baby's weight is measured before and after breastfeeding, and the amount of milk consumption can be calculated by subtracting the two weights. Another measurement technique traces changes in breast volume by computerized imaging of the breast before and after feeding. Another technique provides a volumetric flow sensor inside a silicon nipple cap through which the baby suckles and milk flow data from the sensor is converted into milk volume data which is displayed to the mother. Such systems generally involve attaching electronic or electromechanical flow meters to a fluid flow passage in order to measure the fluid flow. And, yet another conventional technique uses electronic flow measurement modules attached externally to a milk collection device which fits over the mother's breast to measure the milk flow.

Unfortunately, all of the above-mentioned systems are expensive, cumbersome and complex, because of the electronic milk sensing unit, making their suitability for a low-cost, possibly disposable device very unlikely. Also, many mothers would be hesitant to use electronic devices on or near the baby, generally because of their fear of the presence of any electromagnetic radiation. There is also the additional danger that such externally attached parts may become disconnected from the devices and somehow dangerously be ingested by the infant. As the benefits of breast-feeding have become widely known, more mothers are breast-feeding than in the past, highlighting the need for a device and method to indicate a baby's milk intake.

The present disclosure provides a novel device, systems and methods that overcomes the disadvantages of prior art systems and methods.

### SUMMARY

The present disclosure provides a nipple shield adapted to provide a visual indication of a milk flow from a breast to an infant during nursing. The nipple shield has a base unit with a protrusion disposed in a central region of the base unit that is adapted to be positioned over a nipple of the breast and lie securely against the mothers breast. A single channel passageway in a channel cover fluidly extends and connects an inlet opening in a lower surface of the protrusion to an outlet opening on an upper surface of the protrusion. The single channel passageway extends from an inlet opening to a remote location so that all of the milk flow from the inlet opening in the single channel passageway can be visually observed when a mouth of the infant covers a portion of the protrusion. Both the inlet and outlet openings are disposed adjacent to an apex in the protrusion. The channel passageway extends from the apex to a remote location so that the milk flow in the channel passageway can be visually observed when a mouth of the infant covers a portion of the nipple shield.

The nipple shield is also provided to perform multiple tasks to detect various parameters associated with the breast milk flow from the nursing mother. Various connectors can be attached to the nipple shield to detect the various parameters related to the mothers breast milk, the milk flow therethrough, and/or to provide medicines, or the like.

The base unit and the channel cover may be constructed as separate parts and a plasma treatment process may be used to permanently bond the lower cover surface of the channel cover to the upper surface of the base unit to define the channel passageway. Under the plasma treatment process, strong permanent covalent bonds between the lower cover surface of the channel cover and the upper surface of the base unit forms the strong covalent permanent bond between the surfaces.

### BRIEF DESCRIPTION OF THE DRAWINGS

Various exemplary embodiments of this disclosure will be described in detail, wherein like reference numerals refer to identical or similar components or steps The accompanying, and constitute a part of this specification, illustrate embodiments of the subject disclosure and technical date supporting those embodiments, and together with the written description, serve to explain certain principles of the subject disclosure. With reference to the following figures.

FIG. 1 illustrates a partial x-ray view of an exemplary nipple shield for displaying a milk flow from a mother while breast feeding a baby according to an exemplary embodiment of the present subject disclosure.

FIG. 2A is an x-ray plan view of the nipple shield shown in FIG. 1.

FIG. 2B is another x-ray plan view of another nipple shield similar to that of FIG. 1, having an enclosed cavity as an alternative method of indicating the milk flow.

FIG. 2C shows another x-ray plan view of the use of a mechanical protrusion to indicate the milk flow rate by deflecting the milk flow rate in a flow channel.

FIG. 2D shows another x-ray plan view of the nipple shield having an auxiliary container vessel attached to a milk passageway, which fills partly with milk, and then partly empties as the baby sucks and rests.

FIG. 3 shows the addition of a valve in the milk passageway, adapted to improve the milk flow rate detection of the nipple shields shown in FIGS. 1 and 2.

FIG. 4A is a schematic illustration of another exemplary nipple shield incorporating another valve according to the present subject disclosure.

FIG. 4B illustrates another example of a structure and use of another exemplary valve used in the nipple shields having ball valves installed.

FIGS. 4C and 4D shows top views of the exemplary ball valve of FIG. 4B in a closed position and an open position, respectively.

FIG. 4E shows a diaphragm valve, similar to FIG. 4A.

FIG. 4F illustrates an additional strengthening feature of the nipple shield in the form of a circular band in a base adapted to enable the nipple shield to remain latched onto the mother's breast more securely.

FIG. 4G depicts the circular band formed into the nipple shield.

FIG. 5A illustrate a side cross section view of an example of a differential two-way valve construction for use with the nipple shield.

FIG. 5B shows a top perspective view of the differential two-way valve construction shown in FIG. 5A for use with the nipple shield.

FIG. 5C depicts a side cross section view of another example of a differential two-way valve construction for use with the nipple shield.

FIG. 5D shows a top perspective view of the differential two-way valve construction shown in FIG. 5C for use with the nipple shield.

FIG. 5E illustrates a graphical representation of an opening pressure characteristic obtainable from a bidifferential self-actuated differential two-way valve.

FIG. 6A shows an x-ray side view of a medicine dispenser implemented into the nipple shield including shown in FIGS. 1 and 2.

FIG. 6B is an x-ray front view of the medicine dispenser implemented into the nipple shield including shown in FIG. 6B.

FIG. 7 is an upper isometric view of an example of the base unit of a multi-task nipple shield for performing a number of alternative functions related to different aspects of a nursing mother's needs according to another exemplary embodiment of the present subject disclosure.

FIG. 8 is an enlarged view of an outside tip region of a nipple protrusion of the multi-task nipple shield showing various passageways therein shown in FIG. 7.

FIG. 9A illustrates an upper perspective view of the multi-task nipple shield having a cover channel section exploded away and adapted to be attached to a base unit section of the multi-task nipple shield as shown in FIG. 7.

FIG. 9B shows an upper perspective view of a base of the multi-task nipple shield having the cover channel exploded away and adapted to be attached to the channel cover shown in FIG. 9A.

FIG. 10 is a montage showing how the multi-task nipple shield may be used together with various connectors or detection heads to perform various different tasks related to a mother's milk supply.

FIG. 11A illustrates an upper perspective view of the connector attached to the multi-task nipple shield according to another exemplary embodiment of the present subject disclosure.

FIG. 11B shows a side view of the connector attached to the multi-task nipple shield.

FIG. 11C shows a partial top view of a connector attached to the multi-task nipple shield to detect the milk flow rate in the multi-task nipple shield.

FIG. 11D shows a partial top view of another connector having a milk chamber integrated therein and attached to the multi-task nipple shield and adapted to detect the milk flow rate in the multi-task nipple shield.

FIG. 12 shows an upper perspective view of a milk measurement connector attached to the multi-task nipple shield.

FIG. 13 is a perspective view of another embodiment for a nipple shield illustrating a breast milk flow path.

FIG. 14 is a rear view of the nipple shield illustrating the breast milk flow path.

FIG. 15 is a side X-ray view of the nipple shield disposed between a nursing breast and an infant's mouth.

FIG. 16 is a front view of the nipple shield.

FIG. 17 is a rear view of the nipple shield.

FIG. 18 is a top view of the nipple shield.

FIG. 19 is a bottom view of the nipple shield.

FIG. 20 is an exploded view of a channel cover flipped backward and lifted away from a base unit.

FIG. 21 is a cross-section view through the channel cover and base unit of the nipple shield at section A-A in FIG. 16.

FIG. 22 is a cross-section view through the channel cover and base unit at the dome-like protrusion of the nipple shield at section B-B in FIG. 16.

FIG. 23 is an exploded view of another embodiment illustrating a channel cover flipped backward and lifted away from a base unit.

FIG. 24 is a cross-section view through the channel cover and base unit of the nipple shield at section A-A in FIG. 16.

FIG. 25 is a cross-section view through the channel cover and base unit at the dome-like protrusion of the nipple shield at section B-B in FIG. 16.

FIG. 26 shows a schematic of a plasma treatment system used to bond a channel cover to a base unit of a nipple shield.

FIG. 27 illustrates an exemplary evacuation chamber in the plasma treatment system used to bond the channel cover to the base unit of the nipple shield.

FIG. 28 shows an exemplary evacuation chamber initially housed with contamination on the channel cover and the base unit before undergoing a cleaning process by the plasma treatment.

FIG. 29 illustrates the admission of the process gas and ignition of the plasma within the evacuation chamber in the plasma treatment system used to treat the cover surface and the base surface in order to cause the bond of the cover surface to the base surface.

FIG. 30 shows the execution of the plasma treatment on the cover surface of the channel cover, and the base surface of the base unit.

FIG. 31 depicts the ventilation, and removal of the channel cover and the base unit from the evacuation chamber subjected to the plasma treatment.

FIG. 32 shows the removal of contamination and the remaining clean surfaces of the cover surface and the base surface after the plasma treatment.

FIG. 33 illustrates an internet of things (IoT) ecosystem adapted to connect and exchange data between a multi-task nipple shield unit and a network infrastructure.

FIG. 34 details a block diagram of an exemplary smart device adapted for use with the IoT ecosystem.

FIG. 35 details a block diagram of an exemplary server system adapted for use with the IoT ecosystem.

### DETAILED DESCRIPTION

Particular embodiments of the present invention will now be described in greater detail with reference to the figures.

The present disclosure describes an exemplary flexible device or nipple shield 10 for providing a visual indication to a nursing mother as to whether an infant or baby 19a is receiving milk during a breastfeeding session.

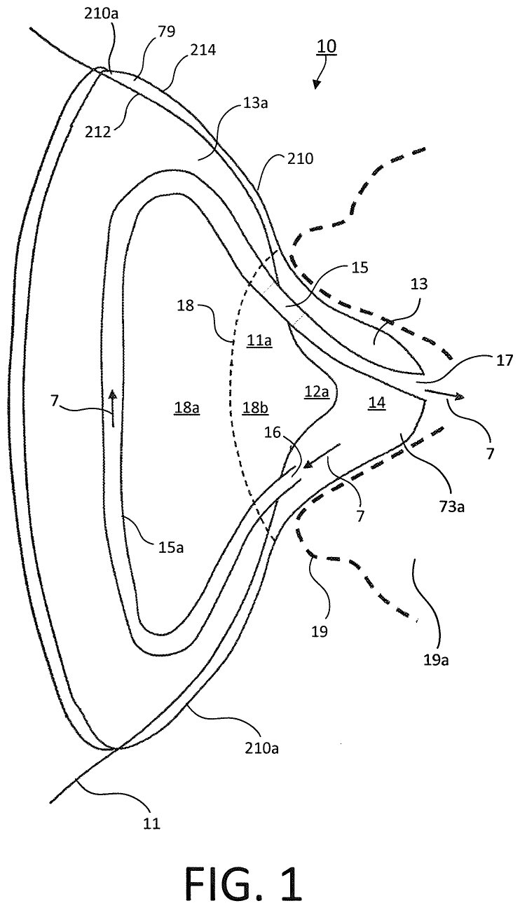

FIG. 1 illustrates a side-elevation and partial x-ray view of a nipple shield 10 constructed according to the present disclosure for visually displaying a milk flow 7 from a mother breast 11 to a feeding mouth 19 of a baby or infant 19a. The nipple shield 10 is fitted over the breast 11 of a woman and has a passageway 15 for directing the milk flow 7 from the nursing woman to the mouth 19 of the infant 19a. During breastfeeding, milk flowing 7 through a viewable portion 15a of the passageway 15 is visible to the mother or to a third party such as a nursing adviser. As shown, the passageway 15 is a single channel passageway connecting an inlet aperture 16 to an outlet aperture 17. Alternatively, presence of the milk flow 7 may be indicated by various methods other than a visual method, such as a color change or audible indication in the nipple shield 10, thereby reassuring the mother that the baby 19a is sucking with sufficient force to receive the milk flow 7 from the mother's breast 11.

A thin body 210 of the nipple shield 10 is typically constructed from a thin flexible material layer 79 and has an inner surface 212 which, when worn, faces and attaches to the breast 11 of the mother, and an outer surface 214 is adapted to face away from the mother's breast 11. During breastfeeding, the outer surface 214 faces, and comes into contact with, the lips and mouth 19 of the baby 19a.

The flexible material of the nipple shield 10 may be a silicone or any other suitable material which is non-absorbent, non-irritating to the skin and sufficiently flexible to be worn comfortably by the mother on her breast 11. Preferably, the nipple shield 10 is form fitting and comfortably wearable over the areola and nipple 12a of the nursing mother. The thin flexible material layer 79 of the nipple shield 10, or at least a part of it, is typically transparent, or semi-transparent so that the milk flow 7 can be visually seen through the viewable portion 15a in the passage 15 of the nipple shield 10 while the infant 19a is nursing. The flexible layer 79 of the nipple shield 10 has a shape and design of a nipple shield, and the flow geometry to cause milk expressed from the mother's breast 11 to flow through the passageway 15. The passageway 15 has a viewable portion 15a disposed in an outer region 18a, which provides a visual indication of milk flow 7. In use, the viewable portion 15a of the passageway 15 in the outer region 18a is disposed outside of the infant's mouth 19 coverage when the infant is feeding. The infant's mouth 19 defines a boundary region 18 (shown in dashed lines) having the outside region 18a of which is in the viewable portion 15a. The viewable portion 15a is within the viewable outside region 18a in which the milk flow 7 is adapted to flow before the milk flow 7 is rerouted through the passageway 15 and supplied back to the central protrusion 13 of the nipple shield 10 from which the infant 19a is sucking. An exemplary distance that the viewable portion 15a of the passageway 15 may be positioned away from the protrusion 13 may be at least 3 centimeters, and/or any suitable distance, so that the passageway 15 is visible outside of the boundary region 18 covered by the mouth 19 of the nursing baby 19a.

The protrusion 13 may also be referred to interchangeably as the device nipple. The protrusion 13 has a dome shape and is adapted to be positioned over the mother's nipple 12a. This protrusion 13 has a geometrical form, when viewed from the outside, has a functional form of a concave region or cavity 14 adapted to receive a nipple 12a of the breast-feeding mother. The thin body 210 of the flexible layer 79 of the nipple shield 10 is constructed to include a base portion 210a with the protrusion 13 disposed in a central region of the base portion 210a. The protrusion 13 defining the cavity 14 over the nipple 12a may be constructed as an integral part of a base portion 210a of the flexible layer, or may be attached in the central region of the base portion 210a. Various methods for attaching the protrusion 13 to the flexible layer are possible, such as for example using a glue, a bonding process, a plasma bonding process and/or any other bonding process capable of fastening the base portion 210a to the flexible layer. The dome-like protrusion may be constructed of another material different from the base flexible layer. Different shaped and different sized nipple shields may be provided to fit an individual mother's breast.

As shown in FIG. 1, the nipple shield 10 when worn covers at least the mother's nipple 12a, and typically at least a large portion of the areola 11a, if not the entire areola 11a, as well. The nipple shield 10 may cover an even larger portion of the breast 11 than the areola 11a. The nipple shield 10 may generally have a circular, or oval shape, although it may have any other shape. The nipple shield 10 has a concave region, also appearing as the protrusion 13 or the device nipple, in accordance with its form when viewed from the outside. This concave region or protrusion 13 is adapted to be positioned over the nipple region adjacent to the nipple 12 of the mother. The nipple region of the mother is understood to mean the mother's nipple 12, although it may include some of the area surrounding the mother's nipple 12 as well. The protrusion 13 is typically formed such that when the nipple shield 10 is worn, there is a hollow space, concave region or cavity 14 formed between the protrusion 13 of the nipple shield 10 and the nipple 12a of the mother. The cavity 14 within the protrusion 13 may be tunnel-shaped. Alternatively, it may be semi-spherically shaped, or may mimic the shape of a mother's nipple 12, as shown in the example of FIG. 1. An outer region 18a of the nipple shield 10 surrounding the dome-like protrusion 13, is adapted to fit conformally over the woman's breast 11, such that when the nipple shield 10 is worn, the outer region 18a remains closely attached to the woman's breast 11 without an appreciable space therebetween. An approximate inner region 18b defined by the boundary region 18 that the infant's mouth 19 will cover between the dome-like protrusion 13 and the beginning of the outer region 18a of the nipple shield 10, as shown schematically in FIG. 1 by the dashed line labeled 18.

The cavity 14 area disposed between the mother's nipple 12a and the protrusion 13 fills with milk 7 from the mother's breast 11. During breastfeeding, the infant sucks on an outer surface 73a of the protrusion 13 that creates a negative pressure in the passageway 15. The negative pressure draws a milk flow 7 into the passageway 15 from the cavity 14 that is filled with milk 7 from the mother's breast 11. The negative pressure generates a vacuum in the passageway 15, assisting in drawing the milk flow 7 from the mother's nipple 12a into the cavity 14 space and into and through the passageway 15 to an outlet or exit aperture 17 to provide the infant 19a with the milk 7. The negative pressure on the nipple shield 10 also forces the thin flexible layer 79 of the nipple shield 10 to adhere to the mother's breast 11, thereby preventing milk from escaping externally from the cavity 14 within the protrusion 13 and from an outer peripheral edge or outer rim of the nipple shield 10. The negative pressure also reduces the entry of air through the outer rim of the nipple shield 10 which would otherwise cause air bubbles in the milk flow 7.

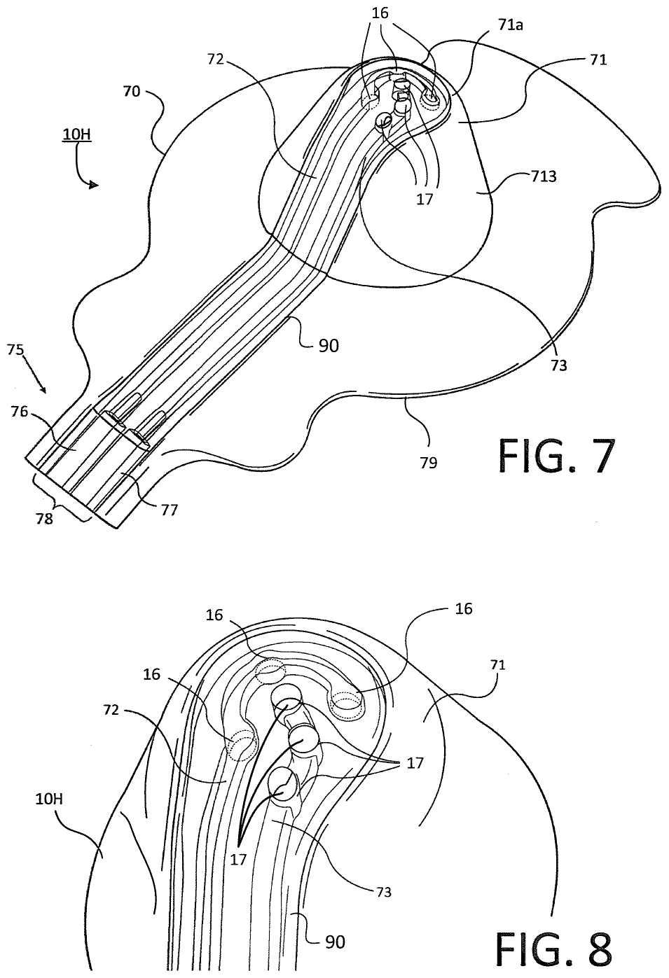

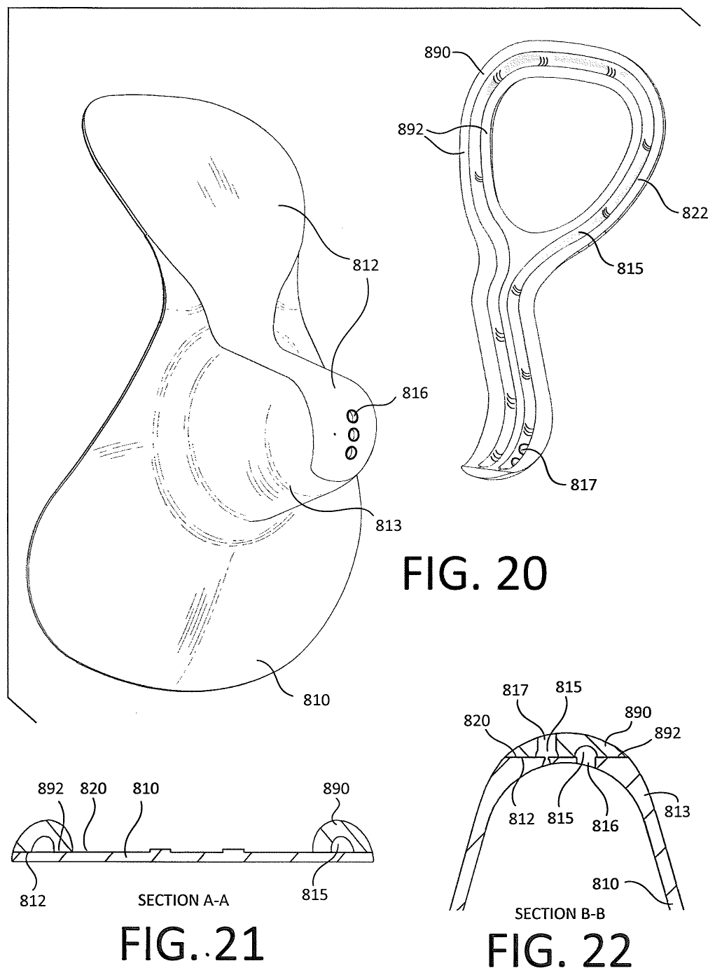

The exemplary implementation of the nipple shield 10 shown in FIG. 1 includes the passageway 15 connecting the cavity 14 with the outer surface 214 at the apex of the dome-like protrusion 13, and routed such that at least a visible portion 15a of the passageway 15 extends outward beyond the infants mouth 19 and is located visibly in the outer region 18a of the nipple shield 10 beyond the boundary region 18. The passageway 15 could be formed within the thin flexible material layer 79 (such as shown in FIGS. 20-25 and described later) of the thin body 210 of the nipple shield 10, or the passageway 15 could be formed on a surface of the thin flexible material layer 79 of the nipple shield 10. Alternatively, the passageway 15 could be a separate section of tubing outside of the thin flexible layer and connected to the nipple shield 10 between the cavity 14 end and the protrusion 13 end. The nipple shield 10 may have one or more passageways, and/or one or more inlet and outlet apertures (such as shown in FIGS. 7-8). The passageways can have a round cross section, or any other suitable shape (such as shown in FIGS. 21-22 and 24-25), for visually displaying the flow of milk with as little resistance as possible.

During breastfeeding, the nipple shield 10 is fitted over the breast 11. The passageway 15 extends from an inlet opening or entrance aperture 16 in communication with the cavity 14, and to an outlet opening or exit aperture 17 at the protrusion 13. The outline of the lips of the baby's mouth 19 sucking on the protrusion 13, are shown in FIG. 1 corresponds to the dashed lines defining the boundary region 18. Negative pressure generated by the sucking actions of the baby 19a on the nipple shield 10, or the mother's physiological reaction to the baby's tongue movements, cause milk to be expressed from the breast 11 of the mother into the cavity 14, from where the milk flow 7 is drawn through passageway 15 to exit the device through exit aperture 17. All of the milk flow 7 supplied to the baby 19a thus flows along the passageway 15, as shown by the milk flow arrows 7 in FIG. 1. The nipple shield 10 may be worn with the entrance aperture 16 of the passageway 15 disposed at the bottom of the cavity 14, such that the milk flow 7 collected by gravity at the bottom of the cavity 14 will be efficiently collected and routed into the entrance aperture 16 of the passageway 15. For this reason, the nipple shield 10 may be constructed with the cavity 14, being the milk collection volume, concentrated at a bottom region of a space between the mother's nipple 12a and the protrusion 13 over the entrance aperture 16 of the passageway 15.

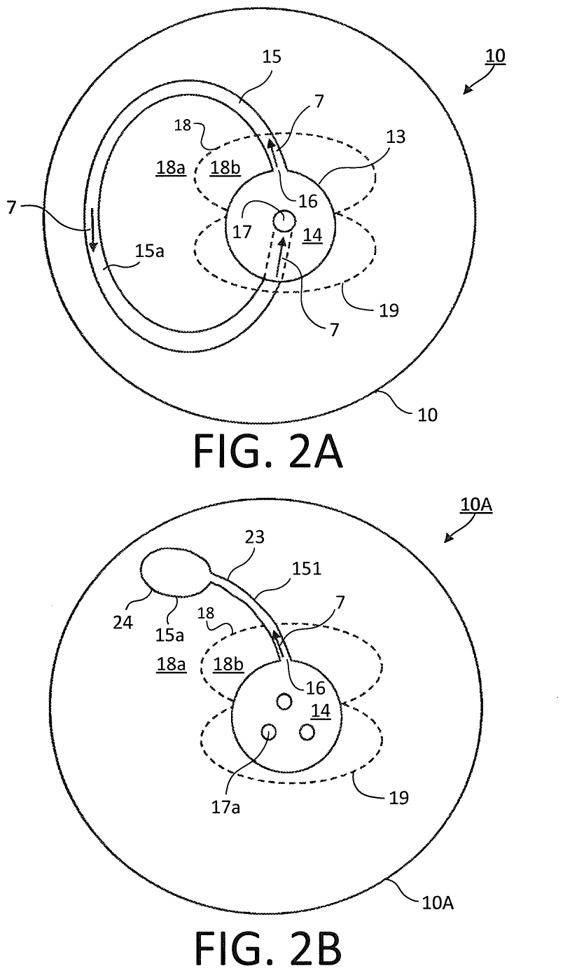

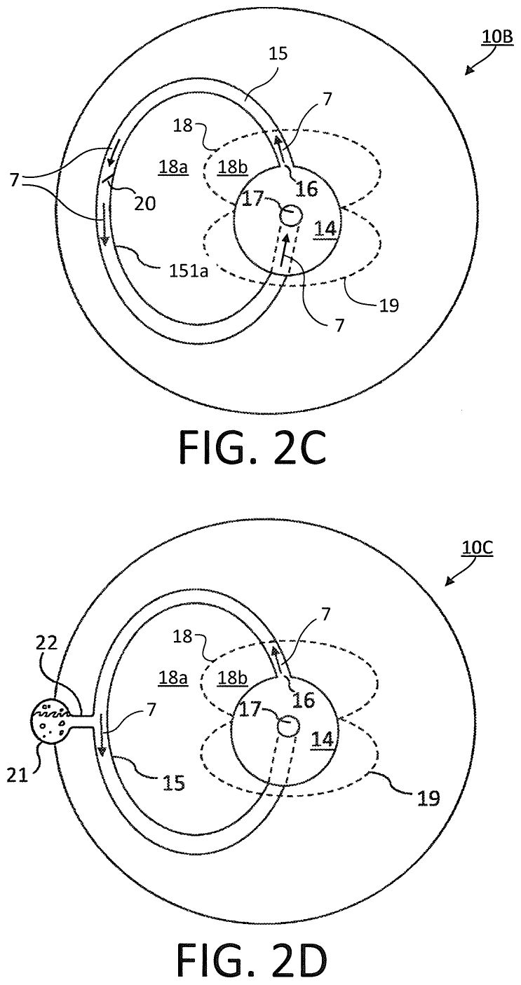

FIG. 2A is a plan view of the nipple shield 10 shown in FIG. 1, illustrating more clearly the course of the milk flow 7 through the passageway 15. The numbering of the features in FIG. 2A are similar to those of FIG. 1. The passageway 15 is routed from the cavity 14 region in the protrusion 13, into and through the inlet or entrance aperture 16 in a radial outward milk flow 7 path outside of the boundary region and into the outer region 18a of the nipple shield 10. The outer region 18a being the space located outside of the boundary region 18 (which is covered by the infant's mouth 19 during feeding) as shown in FIG. 1. By this milk flow 7 routing means, the milk flow 7 passes through the passageway 15 out from the boundary region 18 beneath the lips 19 and mouth of a sucking baby 19a, where it would be hidden from the view of the mother, into the outer region 18a where the milk flow 7 is not visibly obstructed by the lips 19 or mouth of the baby 19a, and where the mother or an assistant can see the milk flow 7 in the viewable portion 15a of the passageway 15. As shown, the passageway 15 may be directed radially outwards towards an outer rim in the outer region 18a of the nipple shield 10, as in the example shown in FIG. 2A. In this outer region 18a, the milk flow 7 can be observed in the viewable portion 15a of the passageway 15. In the milk flow 7 return path from the outer region 18a of the nipple shield 10, and into the nonvisible inner region 18b the passageway 15 re-enters the cavity 14, while maintaining fluid isolation from the entrance aperture 16, and ends at the exit aperture 17 on the outside apex of the protrusion 13, where the milk flow 7 is provided to the baby 19a.

The walls of the passageway 15 may be configured to be transparent or translucent, such that when milk is flowing through the passageway 15, a person viewing the nipple shield 10 observes through the viewable portion 15a a visual indication of whether milk flow 7 is indeed being drawn and/or traveling from the breast 11 and supplied to the baby 19a through the passageway 15. In the case where the flexible material of the nipple shield 10 is not transparent or translucent, at least a portion of the flexible material surrounding the viewable portion of the passageway 15a in an area (such as the outer region 18a) not expected to be obscured by the baby 19a during breastfeeding, may be made of transparent or semi-transparent material. If the passageway 15 is not formed within the flexible material, then the viewable portion 15a or section of the tubing of the passageway 16 itself may be transparent or translucent, to show the milk flow 7 within the passageway 15. As an alternative to visual observation of the milk flow itself through the walls of the passageway 15, the passageway 15, according to other exemplary implementations, can be manufactured of an indicator material, such a material providing a color change when contacted by milk. Another implementation could use a feature or protrusion in the passageway, such as a reed, or a flap, or a paddle-wheel, which generates a sound when the milk 7 flows past or through the sound producing feature audibly indicating the flow of milk 7 therethrough.

Though one method of forming the passageway 15 is by forming it within the flexible layer, the nipple shield 10 can also be constructed using a tube or passageway which either passes outside of the flexible layer of the nipple shield 10, or is attached to its surface, usually the outer surface 214 so as not to interfere with the airtightness of the nipple shield 10 relative to the mother's breast 12. The passageway 15 typically has the shape of a tube, such that it has a generally circular cross-section, although other cross sections may be used without affecting the usefulness of the nipple shield 10.

It is to be understood that the passageway may be constructed as one or more passageways. Likewise, one or more passageways may diverge or converge into one or more inlet apertures 16 and/or outlet apertures 17 respectively in the protrusion 13 region such as shown in FIGS. 7-8. Having multiple inlet apertures and/or outlet apertures will enable the infant to draw in the milk and suck more efficiently since no part of the baby's tongue or lips can then occlude the entire opening having many separate openings, as may be the case with a single outlet opening. Where a plurality of passageways are implemented, at least one of the parallel passageways may extend into an outer region 18a so that the mother can obtain a visible indication of the milk flow 7. For simplification, any number of passageways may be referred to as a single passageway hereinbelow, without intending to limit the application.

FIG. 2B illustrates an alternative structure for a nipple shield 10A in which an indicator passageway 151 does not route the entire milk flow 7 therethrough, but perhaps only a small portion of a milk flow 7 passing through a viewable portion 151a, in order to visibly indicate the presence of a milk flow 7. That is, in this embodiment, a complete passageway from an inlet aperture to an outlet aperture in the protrusion 13 is unnecessary. Instead, by providing a small visual indicating container 15a fluidly connected between the mother's breast and the protrusion 13 of the nipple shield 10A may also provide an indication of the presence of milk 7. In this implementation, the passageway 151 of the nipple shield 10A is a short connection tube or spur 23. The short spur 23 extends from an entrance aperture 16 in the cavity 14, to a small enclosed volume or indicator chamber 24 at a position remote in the outer region 18a distal from the baby and the mother's nipple. The indicator chamber 24 is preferably transparent or translucent, where milk 7 may collect. The actions of the baby between alternatively sucking and relaxing, causes the milk 7 to at least partially enter and exit the closed indicator chamber 24, or to splash about in the indicator chamber 24 under the influence of the surging or alternating pressure fluctuations acting on the milk 7 in the spur 23. The mother can then visually observe the milk 7 entering and leaving the indicator chamber 24 and can thus become informed that the baby 19a is getting his or her milk flow 7. The mother can learn to distinguish between different characteristics of the way in which the milk 7 is splashing about in the indicator chamber 24, or into and out of it, and to relate that appearance to the rate at which the baby 19a is ingesting the milk 7. The actual milk flow 7 from the mother's nipple 12a to the baby's mouth 19, takes place through the outlet aperture 17 in the nipple shield 10A. The interior chamber 24 and spur 23 together can also act as a mechanical protrusion. That is, the milk flow 7 between the interior chamber 24 to the spur 23 may cause the mechanical protrusion to move and change its orientation, thereby indicating the movement of milk flow 7 to an observer.

FIG. 2C shows an additional method for a nipple shield 10B by which the milk flow 7 through the passageway 153 can be indicated. In the embodiment of FIG. 2A, there was mentioned above how a feature or protrusion may be placed in the passageway, such as a reed, or a flap, or a paddle-wheel, could be used as a sound generating element 20, emitting a characteristic sound when the milk flows 7 past or through it. However, in addition to the auditory output generated by such a sound generating element 20, a physical visual indication of the presence of milk flow 7 within the passageway 153 could also be provided. Thus, for instance, as shown schematically in FIG. 2C, a flap is shown as the sound generating element 20 extending into the milk flow 7 path across the passageway 153, is deflected by the milk flow 7 onto its upstream surface, and this deflection may be clearly visible from the outside of the passageway 153 by an observer.

FIG. 2D illustrates yet another embodiment for a nipple shield 10C with an implementation for providing indication of milk flow 7 through a passageway 155. In the implementation shown schematically in FIG. 2D, the passageway 155 comprises a small enclosed container vessel 21 attached to the passageway 155 by means of a short connecting tube 22. Thus, when the milk flow 7 flows past the attached short connecting tube 22, the milk flow 7 surges into the small enclosed container vessel 21, providing a visual indication of the milk flow 7 in the passageway 155. The visual indication is even more strongly emphasized, since the baby 19a draws milk in pulses, during every sucking period, and rests between sucking actions. Consequently, during the sucking period, the milk 7 flowing along the passageway 155 surges into the small enclosed container vessel 21, possibly only partly filling it, and then when the baby relaxes between his/her suction actions, part of the milk 7 in the small enclosed container vessel 21 flows back out into the passageway 155. This constant surge of the milk flow 7 into and out of the small enclosed container vessel 21, possibly including bubbles generated in the surge, provides a good visual indication of the milk flow 7 of the milk 7 as the baby 19a sucks. In FIG. 2D, the small enclosed container vessel 21 is shown oriented horizontal, but it is to be understood that the nipple shield 10C may be used such that the small enclosed container vessel 21 is situated in a vertical and/or any other suitable position, so that the milk flow 7 of the milk is clearly seen filling up and emptying from the small enclosed container vessel 21.

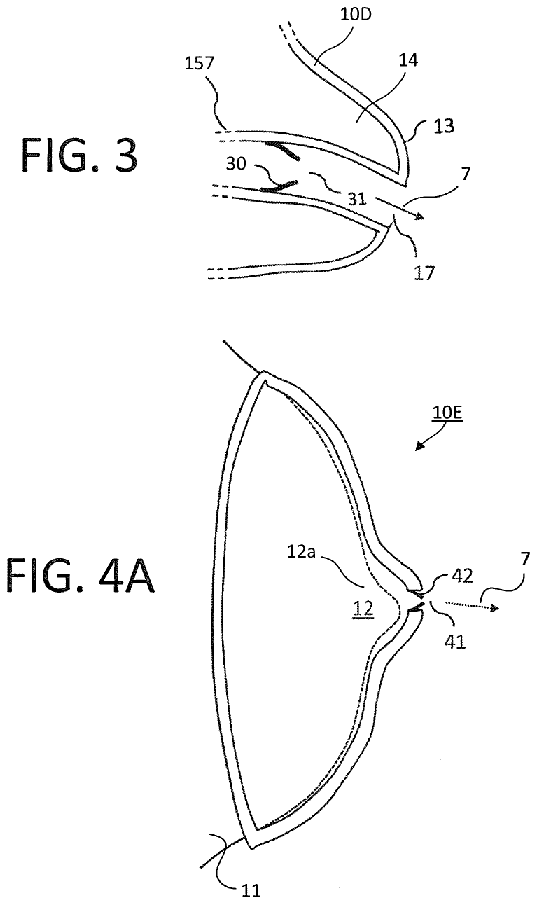

FIG. 3 shows a further embodiment for a nipple shield 10D in which a valve 30 is fitted into a passageway 157, to assist in the maintenance of a sub-pressure in the passageway 157 and the cavity 14 within the protrusion 13, so that the nipple shield 10D remains attached to the mother's breast 11. The valve 30 may be provided to open at a predetermined pressure so that milk flow 7 is enabled through the passageway 157 in a direction from the inlet aperture 16 in the protrusion 13 towards the exit outlet aperture 17 in the apex of the protrusion 13 to the baby 19a.

The valve 30 assists in the maintenance of the negative pressure within the passageway 157, and hence within the cavity 14 within the protrusion 13 when the baby 19a stops sucking. By regulating the negative pressure in the passageway 157, better adherence of the nipple shield 10D to the mother's breast 11 is provided. In addition, the use of the valve 30 decreases the suction effort needed by the baby 19a, since between pauses in the baby's sucking, the baby 19a does not have to generate the entire sub-pressure needed to draw the milk 7 from the cavity 14 within the dome-like protrusion 13. Without the valve 30, the baby 19a would have to generate enough of a sucking force from atmospheric pressure, to draw the milk 7 to the baby's mouth 19. However, with the valve 30, since a predetermined amount of negative or sub-pressure can be held between sucks, the baby 19a can begin sucking from the sub-pressure already maintained in the concave space. The valve 30 thus acts as a flow amplifier, and may reduce the time needed for the feeding session. Additionally, the negative pressure maintained within the cavity 14, may assist the mother by inducing a better milk flow 7 from the mother's breast 11. A baby 19a with more developed sucking abilities may advantageously use a valve 30 opening at a higher absolute pressure. On the other hand, as an alternative consideration, the valve 30 opening pressure can be selected and/or adjusted in order to partly relieve the sub-atmospheric pressure that would be generated by the baby's 19a sucking, so that it does not cause undue discomfort to the mother. The level selected can be chosen according to the mother's level of comfort and acceptance of the negative pressure applied to her nipples 12a. If deemed necessary or advantageous, more than one valve 30 may be used along the passageway 157 to control flow. It is to be understood that reference to opening or closing pressures relate to the absolute pressure difference across the valve 30 at which the valve opens or closes, independently as to whether the valve 30 is situated in a negative pressure region, i.e. in a region of partial vacuum, or in a positive pressure region, i.e. in a region above atmospheric pressure.

FIG. 3 shows a typical example of such an implementation, the valve 30 is shown in this example as a one-way valve in the form of a diaphragm valve or a flap valve which closes when suction is not applied by the baby 19a. In FIG. 3, which is an exploded cross-sectional view of the nipple shield 10D, the flap-valve 30 is shown as a circular diaphragm with a central aperture 31, which opens when a forward milk flow 7 of milk passes through it, but which closes when the milk flow 7 stops due to the baby 19a ceasing to suck, and thus maintains the sub-pressure within the passageway 157. It is to be understood however, that any other form of a valve may also be used, without affecting the functionality of the nipple shield 10D. Similarly, although the valve 30 in FIG. 3 is fitted near the exit 17 of the passageway 157, at the protrusion 13, the valve 30 may equally well be located or formed at any other point in the passageway's 157 path. And, various valve models having various opening pressures can be provided to suit the sucking strength which the baby possesses.

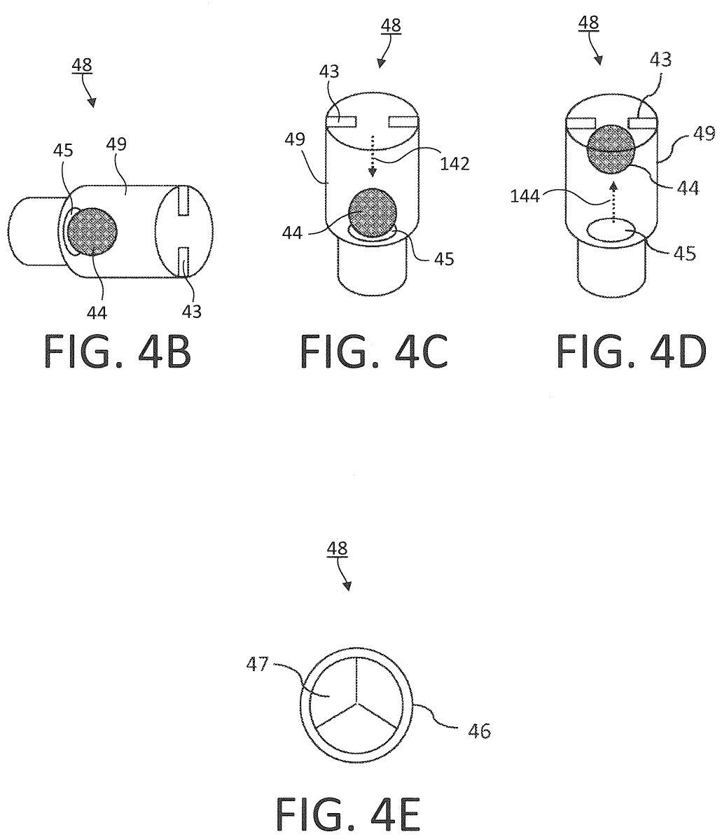

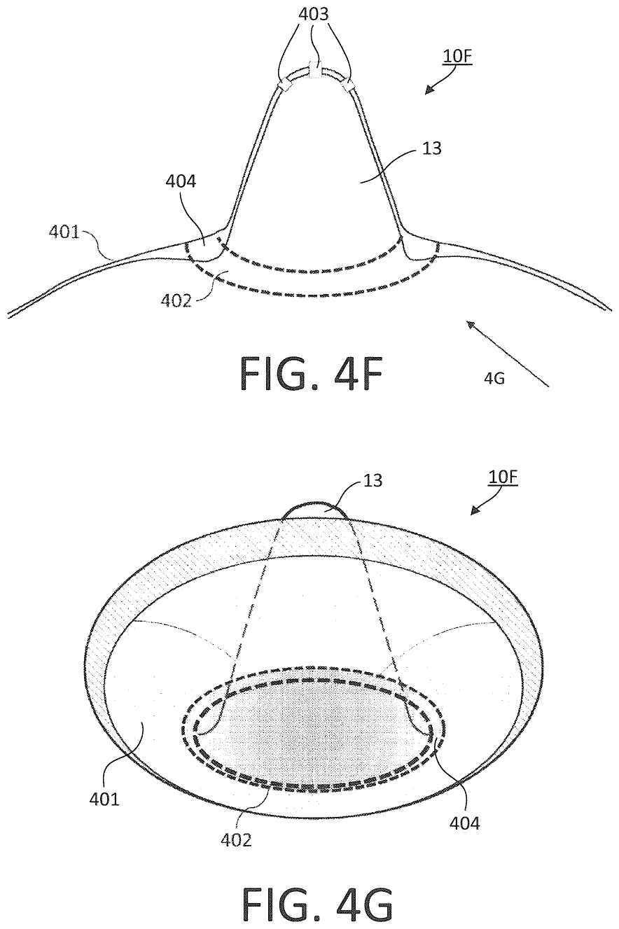

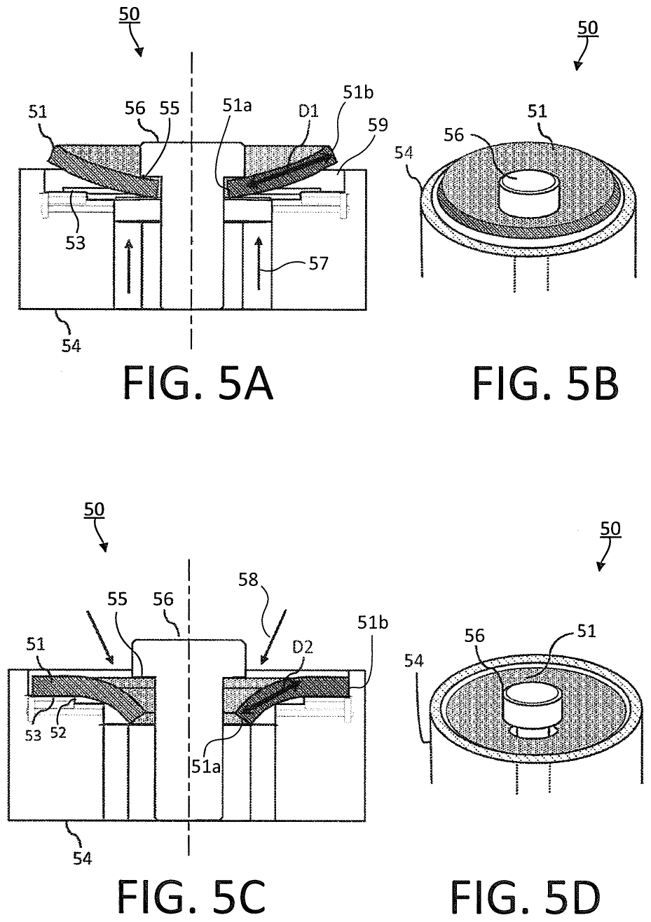

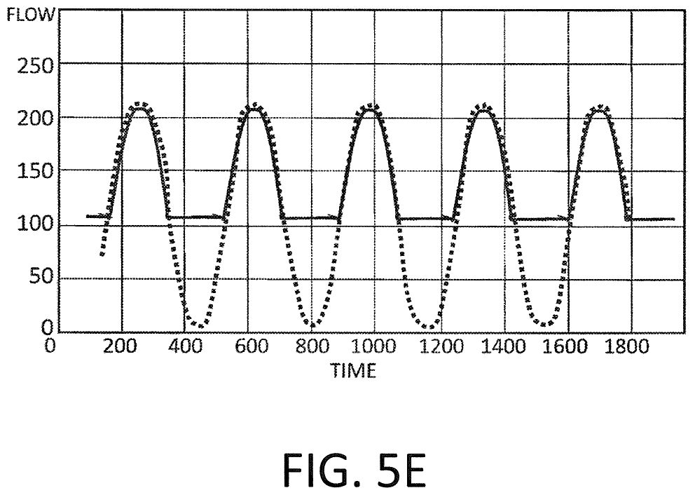

FIG. 4A is a schematic illustration of another nipple shield 10E, constructed according to another exemplary embodiment of the present application. The nipple shield 10E incorporates a valve 42. This improved nipple shield 10E is shown mounted on the nipple 12a of the mother's breast 12 of the mother using it, and the baby 19a receives the mother's milk 7 through the exit opening 41, shown in this drawing as a single exit opening, though there could be more than one exit opening, each with its own valve. When the baby 19a stops sucking, the valve 42 closes, thereby preventing the inflow of air through the exit opening 41 into any space between the nipple shield 10E and the mother's breast 12, thereby assisting in ensuring that the nipple shield 10E remains affixed to the mother's breast 12. The valve 42, is shown as a simple diaphragm or flap valve, but could be any other type of valve which will provide the sealing of the nipple passage 41 required. The use of such a valve in this nipple shield 10E, improves the ease of using the nipple shield 10E, by ensuring latching of the nipple shield 10E onto the mother's breast 12, even when the baby 19a takes a pause from his/her sucking actions. Furthermore, as described in the previous embodiments, and as will be more fully described hereinbelow in connection with FIGS. 5A to 5D, the valve could have a two-level opening pattern, such that in the inflow direction, namely, the flow back in the direction from the baby 19a to the mother's breast 12, the valve could be adapted to require a higher pressure differential to open than a pressure required to keep the valve open in the reverse, outflow direction. This two-way valve allows some inflow of air before closing, to increase the comfort of the mother.

According to a further implementation of the above-described improved nipple shield 10E of FIG. 4A, the incorporation of a flow indicating element (not shown) in the short nipple passage 41 of the nipple shield 10E, enables the nipple shield 10E to provide an indication to the mother that the baby 19a is being provided with a supply of milk 7, about which the mother would not otherwise be certain. The flow indicating element could be a sound producing component such as described above with respect to flap valve sound generating element 20 described in FIG. 2C, or the like, since such a component will provide its indication even if hidden in the mouth 19 of the baby 19a.

FIGS. 4B, 4C, 4D and 4E illustrate schematically other exemplary forms of a valve which could be used in any of the nipple shields of the present subject disclosure, whether a milk flow detection nipple shield, or a nipple shield incorporating valving, as presented in this subject disclosure. FIGS. 4B, 4C AND 4D shows three different views of a ball valve 48, which may be installed either at the protrusion 13 region or some other location of a valved nipple shield of the present disclosure (such as shown in FIG. 4A), or at an exit or somewhere along the length of the milk passageway of the milk flow detection nipple shield of the present disclosure. A ball 44 of this ball valve 48 is able to move along a chamber 49.

As shown in FIGS. 4B and 4C, when the ball 44 sits on an opening aperture 45, which is attached to an internal fluid side of the nipple opening at an exit opening 17, the ball 44 closes the opening aperture 45, and prevents back inflow flow 142 of air or milk 7 into the nipple shield, thereby maintaining a sufficient suction to maintain contact of the nipple shield with the mother's nipple 12a. As shown in FIG. 4D, when the baby 19a sucks on the nipple 12a, the ball 44 within the ball valve 48 is pushed off of the opening aperture 45 by the outward flow 144 of milk 7, thereby enabling the baby 19a to access the flow of milk 7. The construction of the ball valve 48 is configured to prevent the ball 44 from being ingested by the baby 19a. As shown in FIGS. 4B, 4C, 4D, prevention is achieved by a pair of tangs 43, which trap the ball 44 within the cavity 49 of the ball valve 48.

FIG. 4E shows an alternative valve in the form of a simple diaphragm valve 46, similar to that shown in cross-section in FIG. 4A, in which flaps 47 open when a flow of milk 7 occurs outward toward the baby's mouth 19, but returns to the closed position when the flow of milk 7 ceases through the nipple shield. As previously mentioned, any suitable valve can be used for these embodiments.

FIGS. 4F and 4G illustrate a further feature embodied in a nipple shield 10F, in which a band or ring 402 of higher strength is built into a base of the protrusion 13 or device nipple of the nipple shield 10F, such that the nipple shield 10F is held more securely onto the mother's nipple 12a. In FIG. 4F, there is shown a side x-ray view of the protrusion 13 or nipple region of the nipple shield 10F, showing openings 403 in the protrusion 13 for the baby 19a to suck on, and the thin flexible outer region 401 of the nipple shield 10F, for mounting on the mother's breast 12. At a junction 404 of the thin flexible outer region 401 and the dome-like protrusion 13, there is formed a circular band 402 of thicker or less flexible material, which reduces the ability of the nipple shield 10F to bend or to lift off from the mother's nipple 12a and breast 12, thereby assisting in keeping the nipple shield 10F attached to the mother's nipple 12a when the baby 19a ceases sucking. Such a circular band 402 is a strengthening band that can also be applied to any of the milk flow indication nipple shields disclosed herein in accordance with this subject disclosure. FIG. 4G shows a partial isometric lower view of the whole of the nipple shield 10F, viewed from a lower perspective direction marked 4G in FIG. 4F, to show more clearly the position of the strengthening circular band 402. Furthermore, any of the nipple shields described in this disclosure, which incorporate a pressure regulating valve, can benefit from such a strengthening circular band or ring, since maintenance of the vacuum by the valve, when the baby 19a pauses from his/her sucking actions, is enhanced by the use of the above-described strengthening circular band or ring.

FIGS. 5A to 5D show one example of a two-way valve 50, having different opening pressure characteristics in the two flow directions. The two-way valve 50 is operative to avoid excess pressure applied to the mother's nipples 12a from a baby's vigorous sucking. Similarly, the two-way valve 50 is capable of maintaining a predetermined negative pressure within any of the herein described nipple shields in order to keep the nipple shield comfortably attached to the mother's breast. The advantage of the two-way valve is contrasted from a one-way valve in that if a one-way valve was used, when the baby stops sucking, the one-way valve would close and the high sub-pressure generate within the nipple shield would be maintained, thereby continuing to exert the elevated negative pressure on the mother's nipple. This may be uncomfortable to the mother over time. The use of a two-way valve will ensure that when the baby stops sucking, the negative generation of the high negative pressure by the baby within the nipple shield will also cease, and a reverse flow direction of the two-way valve will allow air and/or milk to flow backward into the passageway, but only up to a predefined pressure at which pressure point the two-way valve will be designed to close. Once the two-way valve closes, the sub-pressure at this closing value is maintained but at a negative pressure value less than the sub-pressure generated by the baby, thereby relieving discomfort to the mother but not releasing all of the vacuum, such that the nipple shield remains seated on the mother's nipple.

In more detail, the two-way valve 50 is self-actuated by a pressure difference across the two-way valve 50 in which an opening pressure may be different for two directions of flow. This two-way valve 50 may use a flexible diaphragm 51 disposed across a direction of flow of the fluid. The flexible diaphragm 51 is adapted to have a different flexibility for the two directions of flow. This difference in diaphragm flexibility is generated by providing a different bending length in the flexible diaphragm 51, between a point of support of the flexible diaphragm 51 in the body of the two-way valve 50, and its freely movable inner or outer periphery.

The two-way valve 50 is self-actuated, in the sense that its opening is determined by the differential pressure applied across the two-way valve 50. Such a two-way valve 50 could be used in the nipple shields of the present subject disclosure, and its operation is likewise described in the following paragraphs. It is to be understood however, that the two-way valve 50 could be used in any other application, whether for liquid or for gaseous flow control, and as such, is not intended to be limited to the application described herein, having wide applications throughout industry and medicine.

FIGS. 5A and 5C show cutaway illustrations of the exemplary two-way valve 50, while FIGS. 5B and 5D are upper isometric views of the two-way valve 50. As mentioned hereinabove, such a two-way valve 50 is adapted to enable an essentially free flow 57 of the mother's milk to the baby 19a, while at the same time enabling an inflow 58 of air to the mother's nipple 12a when the baby 19a stops suckling, up to a predetermined negative pressure, in order to reduce the level of the negative pressure acting on the mother's nipples 12a. The two-way valve 50 opening pressure in the outward flow direction 57 should occur at a lower operating pressure than the two-way valve 50 closing pressure in the inward flow 58 direction. By this means, the baby 19a can suckle with minimal obstruction once the two-way valve 50 has opened for the outward flow 57, while the reverse flow 58 for air reducing the level of the negative pressure on the mother's nipple 12a, takes place at a higher differential pressure setting, such that the nipple shield is still held on the mother's breast 12.

In particular, the two-way valve 50 shown in FIGS. 5A to 5D is a two-way differential valve, self-actuated by the pressure difference across the two-way valve 50, and the opening pressure may be different for the two directions of flow. The two-way valve 50 uses a flexible diaphragm 51 which is confined in the valve flow passageway 59, across the direction 57 of the fluid milk flow, between an outer shouldered stepped structure 53 of the valve body 54, and an inner stepped edge 55 of a pedestal 56 mounted centrally in the two-way valve 50, where “inner” and “outer” relate to the radial distances from a central axis of the two-way valve 50. The flexible diaphragm 51 is disposed across the direction of flow of the fluid across the valve flow passageway 59, and is provided with a different flexibility for the two directions of flow 57, 58. This difference of flexibility in the flexible diagram 51 based on the direction of flow 57, 58 across the two-way valve 50 is generated by providing a different bending length of the flexible diaphragm 51, between the point of support of the flexible diaphragm 51 in the valve body 54, and its freely movable within an inner or outer periphery of the flexible diaphragm 51 as shown in FIGS. 5A and 50. The flexible diaphragm 51 is constrained between the inner pedestal 56 having an annular shoulder 55 constraining the inner edge 51a of the flexible diaphragm 51, such that the inner edge 51a of the flexible diaphragm 51 can only bend in one direction (as shown in FIG. 5C), away from the annular shoulder 55 of the pedestal 56. And an outer shoulder 52, 53, of the valve body 54 constraining the outer edge 51b of the flexible diagram 51, such that the outer edge 51b of the flexible diaphragm 51 can only bend in the other direction (as shown in FIG. 5A), upward and away from the outer shoulder 52, 53. Adjustment of the length of the free diameter of the part of the flexible diaphragm 51 that can bend enables control of the pressure across the two-way valve 50 which causes it to open.

As shown in FIGS. 5A and 5B, when the baby 19a sucks and generates a negative pressure, a flow of milk occurs, as indicated by the upwardly directed arrows 57 in FIG. 5A. The terms “upwards” and “downwards” in this connection, relate to the arrow directions shown in the drawing, and have nothing to do with the absolute direction in space. The outer edge 51b of the flexible diaphragm 51 lifts off from its shouldered stepped structure 53, to enable the milk to flow 57 around its outer edge through the passageway 59. The upward movement of the outer edge 51b of the flexible diaphragm 51 is limited by its intrinsic flexibility and by the inner annular shoulder 55 and/or stepped corner of the pedestal 56, around which it bends, and the milk flow takes place around the periphery of the flexed diaphragm 51 through the passageway 59. On the other hand, as shown in FIGS. 5C and 5D, when the baby 19a stops sucking and the milk flow of stops, the negative pressure within the flow passageway 59, generated by the baby's sucking, causes the inner edge 51a of the flexible diaphragm 51 to be pushed inwards and downward by the external air pressure, from its seating position against the annular shoulder 55, thereby bending around in inner edge of the stepped corner 52 of the two-way valve 50. Air, or milk still within the valve passageway 59 or the baby's mouth 19, then enters the two-way valve 50 by flowing between the pedestal 56 and the internally flexed diaphragm 51, as shown by the downwardly directed arrows 58 in FIG. 5C. The inward flow 58 of air/milk continues until the sub-pressure within the nipple shield rises to the level at which the flexible diaphragm 51 closes since the pressure difference across the two-way valve 50 is insufficient to keep the flexible diaphragm 51 open.

The structure of the two-way valve 50 shown in FIGS. 5A through 5D is such that the pressure required to open the flexible diaphragm 51 of the two-way valve 50 can be different for the two directions of flow 57, 58. This may be desirable since the difference in pressure needed to open the two-way valve 50 and enable the flow of milk from the mothers breast 12 to the baby 19a, as indicated by the upward arrows 57 in FIG. 5A, should be low, so as not to place an excess burden on the baby's sucking efforts. This pressure difference should generally be less than the pressure difference at which the two-way valve 50 opens in the reverse direction, in which ingress of air or milk is allowed into the two-way valve 50, as shown by the downward arrows 58 in FIG. 5C, to reduce the level of the vacuum formed within the milk flow passageway 59 and the passages in the nipple shield. This differential opening pressure is achieved by the structure of the two-way valve 50. which is arranged such that the ease of flexing of the flexible diaphragm 51 is different based on the pressure differential in the two directions.

As is known, the bending of a flexible diaphragm is dependent on three factors:
(a) The Young's modulus of the diaphragm material;
(b) The second moment of inertia in the direction of the bending; and
(c) The free diameter of the part of the membrane that bends.

For a specific diaphragm material and shape, factors (a) and (b) are predefined, and therefore, differences in the freedom of bending of the diaphragm is dependent on the free diameter of bending, as will now be explained below.

Referring back to FIG. 5A, when the milk flow causes the flexible diaphragm 51 to flex upward and outwards, the free diameter of bending, or the flexing length, D1, extends from a bending point at a corner of the annular shoulder 55 of the central pedestal 56, to the outer rim 51b of the flexible diaphragm 51. On the other hand, in FIG. 5C, when the inflow 58 of air or milk is desired, and the flexible diaphragm 51 flexes inwards, the flexing length D2 extends from bending points at the corner 52 of the stepped shoulder structure 53 of the valve body 54, to the inner rim 51a of the flexible diaphragm 51. Since the bending length D1 for the outward flow 57 direction of FIG. 5A is longer than the bending length D2 for the inward flow 58 direction of FIG. 5C, the resistance to bending the flexible diaphragm 51 upward (in FIG. 5A) is less and easier to bend for the outward flow 57. The resistance required to bend the flexible diaphragm 51 downward (in FIG. 5C) is higher and harder to bend for the inward flow 58 That is, the flexible diaphragm 51 of the two-way valve 50 opens and closes at a lower pressure difference in the outward flow direction (FIG. 5A) than in the inward flow direction (FIG. 5C). It is to be understood though, that depending on the specific situation for which the two-way valve 50 is designed, the opening pressures could be arranged to be equal or even reversed, with the specific values determined by the selected flexing or modifying the lengths, D1 and D2.

Reference is now made to FIG. 5E which is an exemplary graph showing the opening pressure characteristics obtainable from a bidirectional, self-actuated differential valve, as shown in FIGS. 5A to 5D. In the graph, there is shown the flow conduction characteristics of the valve in the two opposite directions, one being shown by the dotted line and the other by the solid line. The abscissa (X axis or TIME) of the graph shows the elapsed time in nominal units, while the ordinate (Y axis or FLOW) shows a valve flow conduction characteristic, also in nominal units. The pressure is applied to the valve according to a sinusoidal periodic characteristic. In the direction of easier flow (57, FIG. 5A), namely the direction in which the valve remains open to a lower differential pressure across the valve, this characteristic being shown by the dotted line, the flow ranges from 0 to 200 units as the pressure rises and falls from its minimum to maximum value. In the direction of more restricted flow (58, FIG. 5C), namely the direction in which a higher differential pressure is required to open the valve, this characteristic being shown by the full line, the valve remains shut until the pressure reaches a value when it begins to provide a flow at the 100-unit level, and remains open right up to the 200 unit level.

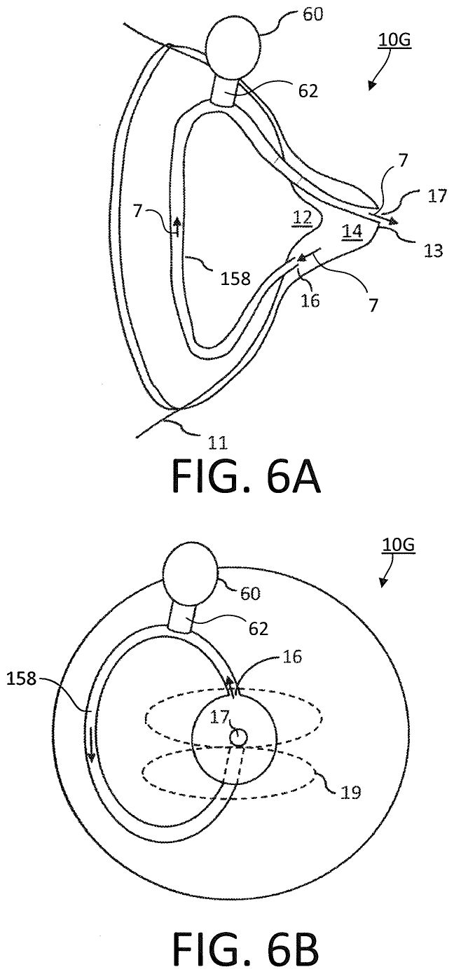

FIGS. 6A and 6B illustrate schematically another exemplary nipple shield 10G having a medicine dispenser implementation for the nipple shield of the type shown in FIGS. 1 and 2. As known, it is challenging to administer medicine to infants, and the amount of medicine given should be accurate. The conventional use of a spoon or a syringe to feed a baby medicine is unreliable and inaccurate. It is likewise difficult to get the baby to ingest the dose since it is very likely that the baby may spit out part, or all, of the medicine. FIG. 6A shows an x-ray sectional/isometric view of the nipple shield 10G, while FIG. 6B shows a plan view of the nipple shield 10G. According to this feature of this nipple shield 10G, a medicine container reservoir 60 is fluidly connected to the milk passageway 158 by means of a connecting tube 62. The medicine container reservoir 60 filled with a correct quantity of a medication to be administered to the infant and is attached to the passageway 158 such that the medication is dispersed from the container reservoir 60 and mixed in the milk flow 7 within the passageway 158 in a controlled manner. In use, the infant will receive all of the measured medicine that is mixed in with the milk from the mother during breast feeding. The medicine container reservoir 60 may be removable detachable for multiple reuses and easy refilling. Various factors will affect the mixing rate of the medicine with the milk flow 7, such as depending on a flow rate of the medicine from the medicine container reservoir 60 to the milk passageway 158.

This flow rate of the medicine can be controlled either by a valve, or it can be determined by the fluid resistance of the connecting tube 62, according to a cross sectional area and the length of the connecting tube 62, and by the viscosity of the medicament. The timing of the addition of the medicine to the baby's milk can be determined either by a stop-valve in the connecting tube 62, or by an air inlet valve in the top of the medicine container reservoir 60. The baby 19a will thus receive all of the medicine dose, or any other fluid which it is desired to provide to the baby 19a with, mixed with the mother's milk during breast feeding, and at the flow rate desired, such that even the taste of a medicine would be masked. The medicine container reservoir 60 is shown schematically as a balloon shaped volume, but it is to be understood that it could have any suitable form for this purpose, such as a prefilled vial, or a container with a closable lid. The medicine container reservoir 60 may be detachable for easy refilling, or it may be attached during manufacture to the nipple shield 10G, as part of a single use disposable device.

FIG. 7 is a schematic external isometric view of another example a nipple shield 10H configured as a multi-task nipple shield of the present disclosure. The nipple shield 10H can be used for performing a number of alternative functions related to various aspects of a nursing mother's needs. The nipple shield 10H includes a base unit 70 and a cover channel 90. The base unit 70 has a thin flexible layer 79, comparable in thickness to the other nipple shields described herein, and is adapted to be fitted by the mother over her breast 11. The nipple shield 10H has a central region or dome-like protrusion 713 that defines a cavity 14 for a protruded nipple volume 71, which is adapted to fit over the mother's nipple 12a.

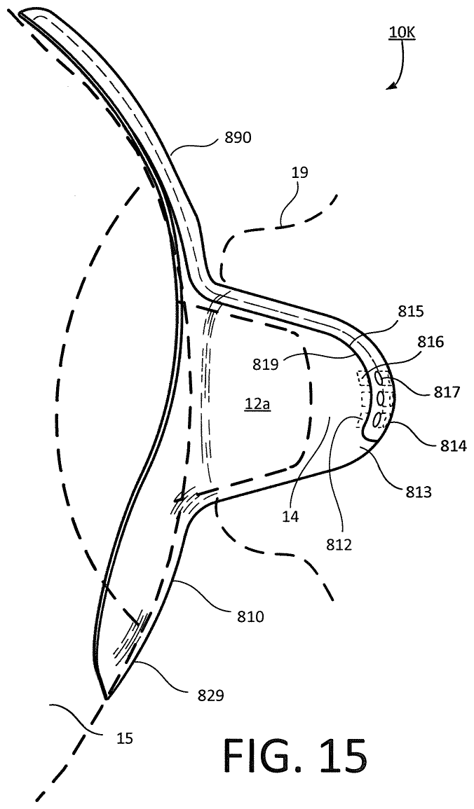

The nipple shield 10H is adapted to transfer the mother's milk 7 from her nipple 12a to the baby's mouth 19 by passageways which conveys the milk 7 to and from a location remote from the nipple 12a. That is, the nipple shield 10H incorporates a pair of passageways, an inlet passageway 72, and an outlet passageway 73. The inlet passageway 72 has a plurality of fluidly connecting inlet apertures 16. The outlet passageway 73 has a plurality of fluidly connecting outlet apertures 17. The passageways 72, 73 are embedded within the thin flexible layer 79 of the channel cover 90 of the nipple shield 10H, each of the inlet and outlet passageways 72, 73 fluidly connecting a tip 71a of the protrusion 713 to a remote location disposed in a remote region 75 which will be accessible to the mother or an assistant while the baby 19a is sucking on the nipple or dome-like protrusion 713 of the nipple shield 10H. The inlet passageway 72 shows a plurality of inlet apertures 16 fluidly connected and opened to an inside surface (819, best shown in FIG. 15) of the protrusion 713 over the protruded nipple volume 71. The other embedded plurality of outlet apertures 17 of the outlet passageway 73 are fluidly connected and opened to an outside surface (814, best shown in FIG. 15) of the protrusion 713 over the protruded nipple volume 71. The first of these inlet apertures 16 is adapted to convey milk from the inside surface of the dome-like protrusion 713 over the protruded nipple volume 71 where inlet passageway 72 is in direct contact with the milk expelled from the mother's nipple 12a, to the remote region 75 remote from the dome-like protrusion 713. The other of these outlet passageways 73 is adapted to convey the milk from the remote region 75 back to the outside surface of the dome-like protrusion 713 over the protruded nipple volume 71 where the outlet passageway 73 can supply the milk to the baby 19a sucking on the outside surface of the dome-like protrusion 713 of the nipple shield 10H.

At the remote region 75, each of the passageways 72, 73 terminates in a fluid connection pole 76, 77 respectively, at a fluid connector port 78. The two poles 76, 77 having predetermined known physical dimensions, and being disposed at a predetermined known distance apart. Although the simplest and most cost-effective configuration is the use of a pair of passageways 72, 73, the nipple shield 10H could also be constructed using more than a pair of inlet and outlet passageways, so long as at least one of the passageways is an inlet passageway that is connected from an outer surface of the dome-like protrusion 713 to a fluid connection element at a remote region, and at least another of the passageways is an outlet passageway that is connected from an inner surface of the dome-like protrusion 713 to a fluid connection element to a remote region in at a remote location. Although the nipple shield 10H shown is made of a single construction, it is to be understood that the nipple shield may be made from various components, and secured together into a single nipple shield assembly.

At this remote region 75, the fluid connection port 78 can receive any of a number of different operational heads or connectors 100. The connectors 100 can be removably or permanently attached to the fluid connection port 78. The fluid connection port 78, and the various operational connectors 100 may have matching standardized fluid flow connections that may be attached to the remote connectors 100 on the base unit 70 of the nipple shield 10H. According to an aspect of this subject disclosure, the connection port 78 can be universal to receive various different types of connectors 100.

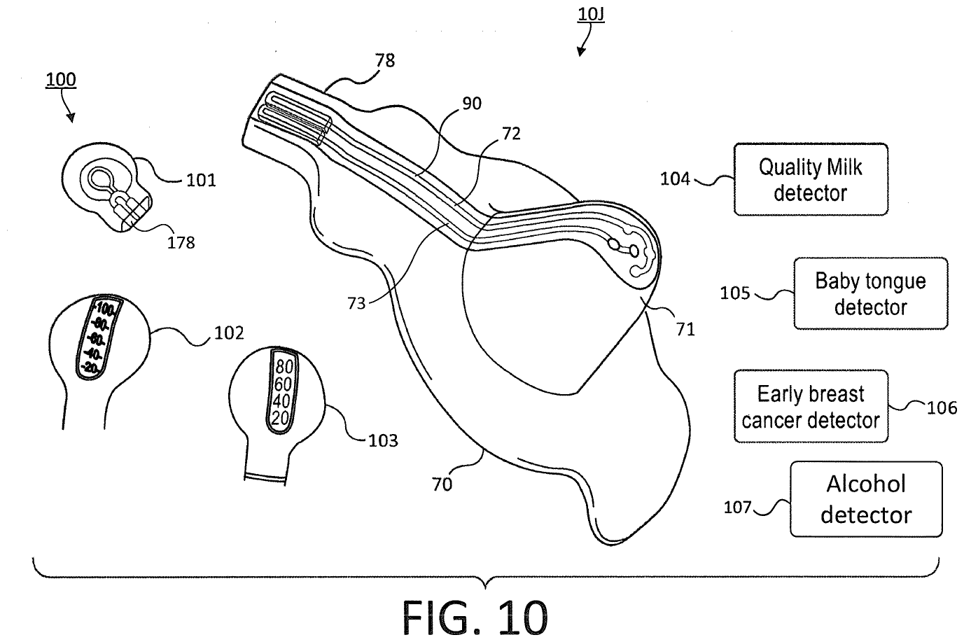

Each of the various connectors is adapted to perform its own dedicated function as it related to the milk and/or milk flow. The number of different operational connectors 100 may be adapted for use with the nipple shield 10H to collect and provide various information to a user. Each connector 100 being adapted to perform a separate function related to the milk or the milk flow The connectors 100 can be attached to the fluid connector port 78 as will be further shown and described in FIG. 10 below. As shown in FIG. 7, the nipple shield 600 has various universal uses, and the particular use made of the nipple shield 600 depends on the connector 100 attached to the remote fluid connection port 78 of the nipple shield 10H. The various connectors 100 can be attached for various measurement or indicational features and/or functionality. Examples, include but are not limited to, flow measurement, flow indication, medicine addition, measurement of the suction pattern of the baby, milk quality analysis, detection of markers in the milk indicating illness of the mother, and numerous other functional uses according to this subject disclosure.

FIG. 8 is an enlarged outer view of the dome-like protrusion 713 of the nipple shield 600 of FIG. 7, showing how the inlet passageway 72 is connected to the inlet apertures 16 on an inside surface of the dome-like protrusion 713 over the protruded nipple volume 71, and how the outlet apertures 17 of the outlet passageway 73 is connected to the outside surface of the dome-like protrusion 713 over the protruded nipple volume 71. As shown in FIG. 8, inlet apertures 16 of the first inlet passageway 72 are open to the inside surface of the dome-like protrusion 713, while the outlet apertures 17 of the second passageway 73 are open to the outside surface of the dome-like protrusion 713. Three inlet apertures 16 are shown in the inlet passageway 72, and three outlet apertures 17 are shown in the outlet passageway 73, in order to provide a low resistance to the fluid flow, though it is to be understood that this is simply an exemplary implementation, and that any other number and form of the apertures that provide a suitable flow of milk may be used.

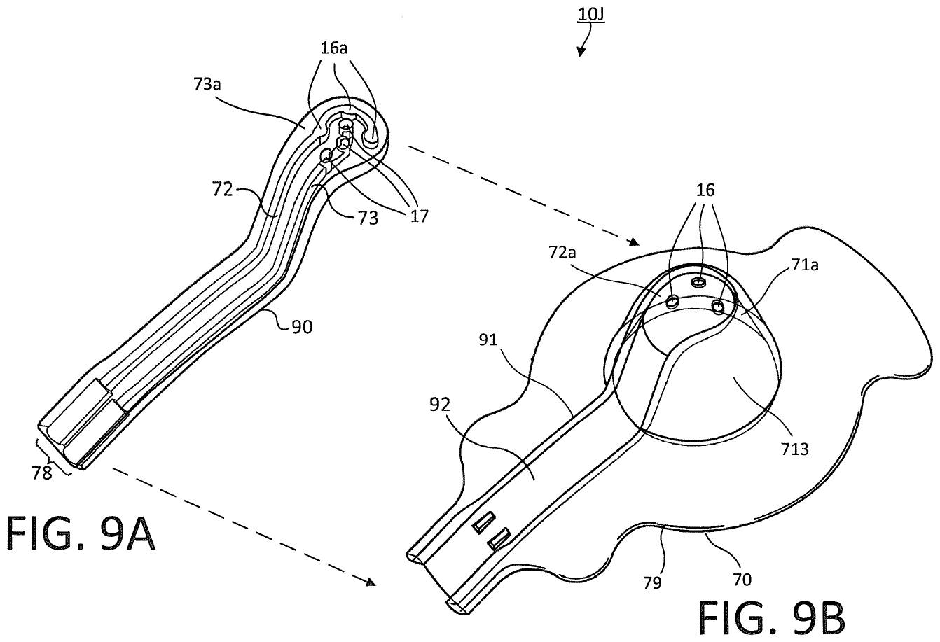

FIGS. 9A and 9B illustrates a method of facilitating the manufacture of the nipple shield 10J as a two-piece nipple shield construction. As shown, the nipple shield 10J has a channel cover 90 that is adapted to be attached to a base unit 70 to resemble the nipple shield 10H construction shown in FIG. 7. Since the properties required of the separable channel cover 90 of the nipple shield 10J containing the inlet and outlet passageways 72, 73 may be different from those required of the thin flexible layer of the nipple shield device 10H shown in FIG. 7, it is advantageous to manufacture the channel cover 90 separately from that of the base unit 70 itself. FIG. 9A shows the channel cover 90 as a strip section of material containing the inlet and outlet apertures 16, 17 and the inlet and outlet passageways 72, 73 molded within the channel cover 90, with a fluid connection port 78 disposed at a remote extremity, mimicking the remote location shown in FIG. 7. FIG. 9B shows a thin flexible body 91 of the nipple shield 10J, with a shallow channel 92 formed within part of the thickness of the thin flexible body 91 of the base unit 70, having an elongated shape adapted to receive the channel cover 90 containing the inlet and outlet passageways 72, 73 molded within the channel cover 90. The tip 71a of the dome-like protrusion 713 of the nipple shield 10J has inlet apertures 16 formed therein connecting to an inner surface 72a of the dome-like protrusion 713 The position of the inlet apertures 16 on the dome-like protrusion 713 matching the inlet aperture pockets 16a in the inlet passageway 72 in the channel cover 90 and adapted to convey milk from the inside of the protruded nipple volume 71 towards the fluid port 78 at the remote location.

FIG. 10 illustrates a montage of connectors 100 surrounding the nipple shield 10J assembled and adapted for multiple tasks. In particular, FIG. 10 shows the base unit 70 securely attached to the channel cover 90 as a single nipple shield 10J assembly. The nipple shield 10J is a a multi-task nipple shield of the present disclosure and is used together with the various connectors 100 available for various different tasks related to the mother's milk supply. As shown, the connectors 100 have a female connection 178 that is adapted to plug into the fluid connection port 78, located remotely from the dome-like protrusion 713 of the nipple shield 10J. A number of different connectors 100, in particular different detection heads 101, 102, 103, 104, 105, 106 are shown in FIG. 10. All of the connectors 100 have one feature in common, namely that the connection of the connector 100 to the fluid connection port 78 completes the fluid circuit for the milk between the inlet and outlet passageways 72 and 73. As such, the baby 19a can freely suck milk from the mother's nipple, with the milk flowing 7 along the inlet passageway 72 to the connector 100 attached at the fluid port 78, and then the milk flows 7 back through the outlet passageway 73 to the baby's mouth 19. The various connectors 100 can be made for single or multiple use.

As mentioned, various connectors 100 providing a variety of different uses are possible and will be described herein. A flow detection connector 101 is provided to detect a milk flow in the nipple shield 10J. The flow detection connector 101 provides an indication to the mother that the baby 19a is receiving a flow of milk through the nipple shield 10J. The flow detection connector 101 can use any of the flow indicating features described in the earlier implementations of a nipple shield of the present disclosure.

A flow measurement connector 102 may be provided to make quantitative measurements of the quantity of milk flowing from the mother to the baby 19a. The flow measurement connector 102 can make measurements based on the technique described in U.S. Pat. No. 7,896,835 for “Apparatus and Method for Measuring Fluid Flow to a Suckling Baby”, commonly owned by the present applicant, in which a fraction of the main milk flow is passed through a measurement channel, where the quantity of milk collected in the measurement channel provides a measure of the milk drawn through the main channel. Alternatively, the flow measurement connector 102 can incorporate a micro-technology flow sensor in a loop formed in the flow measurement connector 102, through which the main milk stream flows to make appropriate measurement detections. An output signal can be generated based on the detected measurement and transmitted to a remote reader that displays the flow rate, and can integrate the flow rate to provide the quantity of milk delivered. A transmission system that communicates with, for instance, a smartphone application, would be advantageous.

A medication delivery connector 103 for the addition of medication to the baby's milk feed, can be incorporated as a small medication enclosure connected by a channel to the milk passageway (such as shown in FIGS. 6A and 6B), such that the medication can be slowly added to the milk flow of the baby's milk. In addition to the functionalities of the nipple shields previously described in this disclosure, the use of a universal multitask nipple shield, enables determination of a number of additional measurements and features.

A milk quality connector 104 which is a miniature chemical or spectroscopic analyzer. The milk quality connector 104 can enable the determination of the quality of the milk or of its various components such as its fat level, or the insecticide content, and similar analyses.

A suction connector 105 may be used for determining the sucking efficiency of the baby 19a, such as by measuring the level of vacuum or negative pressure generated within the suction connector 105, or the length of a sucking period compared with a rest period of the baby, or other features characterizing the baby's sucking ability.

A disease detection connector 106 may be provided to analyze the mother's milk in a disease detection connector 106, which would include a micro-spectrometric or bio-chemical analysis unit, to provide advance warning of an illness or disease, which can manifest itself in the baby's milk delivered from the mother's breast. The disease detection connector 106 may have the potential of early detection of breast cancer of the mother using the device. An advantage of the nipple shield 10J of FIG. 10, is that all of the above suggested functions can be executed with their relevant connector attachments, without interfering in the milk flow 7 provided to the baby.

An alcohol detection connector 107 is provided to determine an alcohol content level in a mother's milk. Since it is possible that a mother may enjoy an alcoholic beverage from time to time, there is a need to provide a notification to the mother that an acceptable level for breast feeding has been restored in the milk 7 after drinking the alcoholic beverage. Since the effects of maternal alcohol (ethanol) ingestion during lactation are complex and depend on the pattern of maternal drinking. It is known that alcohol decreases milk production and disrupts nursing until maternal alcohol levels decrease. Breastmilk alcohol levels closely parallel blood alcohol levels. The highest alcohol levels in milk typically occurs 30 to 60 minutes after an alcoholic beverage is consumed, but food delays the time of peak milk alcohol levels. Therefore, nursing after 1 or 2 drinks can decrease the infant's milk intake by 20 to 23% and cause infant agitation and poor sleep patterns. The alcohol detection connector 107 can provide safe detection levels and an indication to a mother as to when they may restart breast feeding to the infant 19a.

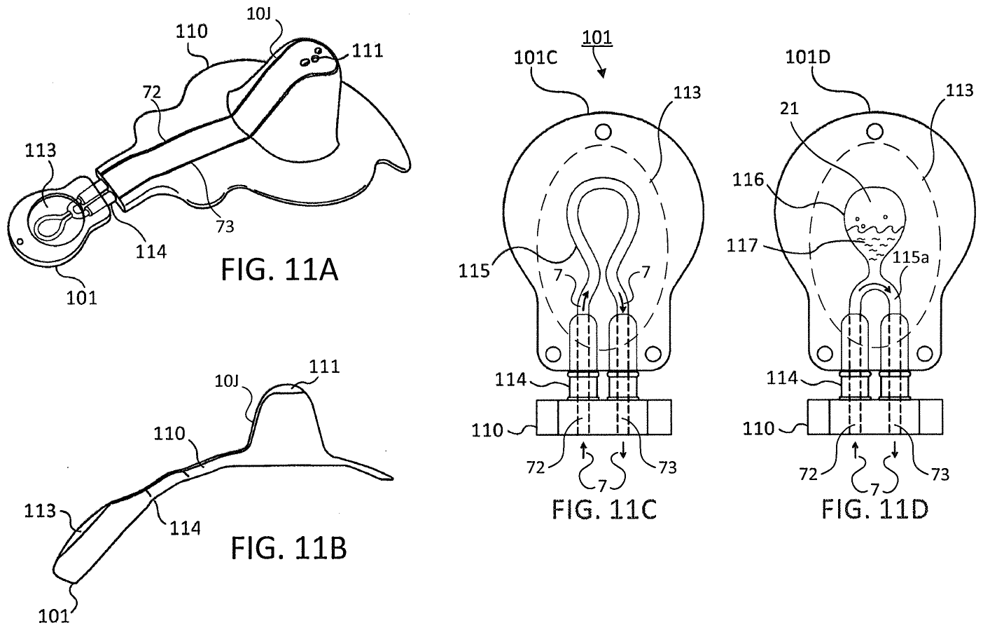

FIGS. 11A to 11D illustrate in greater detail, an exemplary use of the nipple shield 11J of FIGS. 7 to 10 in combination with a flow indication application using the flow detection connector 101 101 of FIG. 10. In FIG. 11A, there is shown the base unit 110 of the nipple shield 10J, with the nipple openings 111 in the apex region of the dome-like protrusion 713, and with the flow detection connector 101 attached at the fluid port connection 114. The flow detection connector 101 is shown having a viewing window 113, which should be transparent in order to facilitate viewing of the milk flow indication within the attached flow detection connector 101. FIG. 11B shows a side view of the base unit 110 attached to the flow detection connector 101, to show the orientation in which the transparent viewing window 113 is positioned remotely from the nipple openings 111, so that the milk flow can be readily viewed through the viewing window 113 by a nursing mother or an assistant.

FIGS. 11C and 11D schematically show two alternative schematic implementations of the flow detection connector 101, on a larger scale, in order to show the details of the structure of the flow detection connector 101. The two different figures show alternate ways in which the indication of the milk flow can be generated.

In FIG. 11C, there is shown a flow detection connector 101C, which uses the method of indicating the flow as shown in FIG. 2A. That is, the milk flow 7 of milk coming through the inlet passageway 72 in the base unit 110 from the protruded nipple volume 71 within the dome-like protrusion, is directed around a transparent or translucent loop of tubing passageway 115 within the viewing window 113, and then the milk flow 7 passes back out of the viewing window 113 within the flow detection connector 101C into the outlet passageway 73 of the base unit 110 for returning to the nipple openings 111 on which the baby 19a can suck. The mother can thus have a direct view of the milk flow 7 within the viewing window 113.

FIG. 11D illustrates an alternative flow detection connector 101D further implementing the construction shown in FIG. 2D. In this implementation, a visual indicating milk chamber 116 is provided in the flow detection connector 101D and is fluidly connected at the fluid port connection 114 to the inlet and outlet passageways 72, 73 to convey the milk flow 7 from the base unit 110, and back to the base unit 110, adjacent to the fluid port connection 114 within the indicating head unit 101D. As the milk flows 7 through the passageway 115a in the flow detection connector 101D, part of the milk flow 7 enters a closed milk chamber 116, where the milk 117 can be viewed through the viewing window 113 providing a visual indication of the milk flow 7 of the milk 117 in the passageway 115a. In particular, as the baby 19a repeatedly sucks on the mother's breast 12, the milk 117 passes down the passageway 115a in spurts, and enters the milk chamber 116 in surges synchronized with the sucking actions of the baby 19a. These constant surges of milk 117 into and out of the milk chamber 116, provide a good visual indication of the milk flow 7 of the milk 117 as the baby 19a sucks.

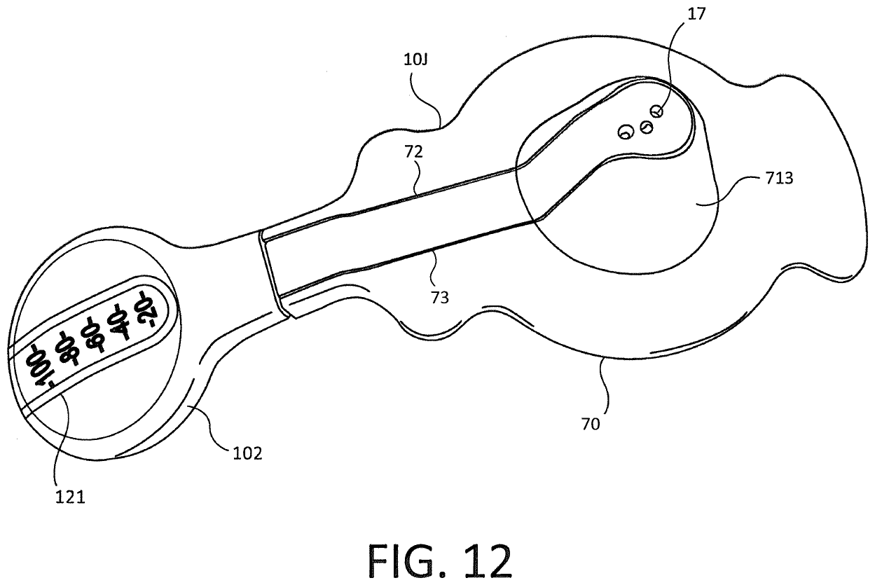

FIG. 12 shows an external perspective view of a schematic implementation of the flow measurement connector 102 connected to the base unit 70 of the nipple shield 10J. In the alternative to the milk flow processes described above, the flow measurement connector 102 may operate using the same inventive concept as is described in the above referenced U.S. Pat. No. 7,896,835. That is, the structure of a flow measurement connector comprises two flow paths (not shown in FIG. 12) for the milk flow. In a first milk flow path, a main passageway is provided for milk flow directly to the baby, having a substantially lower resistance to the fluid flow of the milk than the second flow path through a second passageway. A second flow path is connected in parallel to the first flow path and has a substantially higher resistance to the milk flow than the first flow path. Consequently, the milk flows into the second flow path at a substantially slower rate than in the main path, and the position to which the advancing front surface of the milk reaches, is a measure of the total quantity of milk that has passed through the flow measurement connector 102. The position of the front of the milk fill can be determined against graduations 121 on the flow measurement connector 102, which can be calibrated according to a total quantity of milk taken by the baby 19a. As an alternative implementation, use can be made of a micro-flow meter for measuring the flow rate of milk flow in the flow measurement connector 102, with a measurement chip for providing an output signal of the flow rate, or by integrating the flow rate, the total quantity of milk consumed by the baby.

Another advantage of the nipple shield structure of this subject disclosure is that it can be produced at low cost by conventional polymer production techniques, without the need for any additional mechanical or electronic components. This construction is beneficial in that it enables the nipple shields to be constructed as unitary and compact, self-contained devices, of sufficiently low manufacturing cost for single use, disposable milk flow indication devices. This is a significant benefit over previously described nipple shields that required cumbersome mechanical or electronic devices. This construction also provides for a more universal use of a milk indicating device for the nursing mother. Furthermore, the construction as a unitary integral nipple shield avoids the danger of external parts becoming detached with the fear that the nursing infant may ingest and/or choke on such a detached part. Additionally, an electronics-less device capable of providing information about milk flow to the baby, constructed of inert polymer materials, will also allay fears of a mother about the operation of electronic devices so close to a young nursing infant. It is also an aspect of this subject disclosure to construct the nipple shields from various parts and assembled and attached to each other as an integrated assembly.

Although the nipple shield description above refers to use with a mother of an infant supplying milk, it is to be understood that this concept is not intended to be limited only to a mother using the device to breast-feed her baby and its technology has many other applications. For example, the visual observation of the nipple shield technology may be adapted for use being mounted to a bottle. As a visual observation device attached to a nursing bottle, direct evidence of the milk flow is obtained while the baby is feeding, rather than interrupting the feeding session to hold the bottle upright to read the milk level on the graduations of the bottle. This is beneficial where a bottle is opaque and milk flow cannot be readily seen therethrough.

It is to be understood that this disclosure contemplates the need to clean the various nipple shields and components using a brush, a machine and/or any other suitable means for washing and sterilizing the components associated with this subject disclosure, such as a water sterilizer system, by steam and/or boiling, etc.

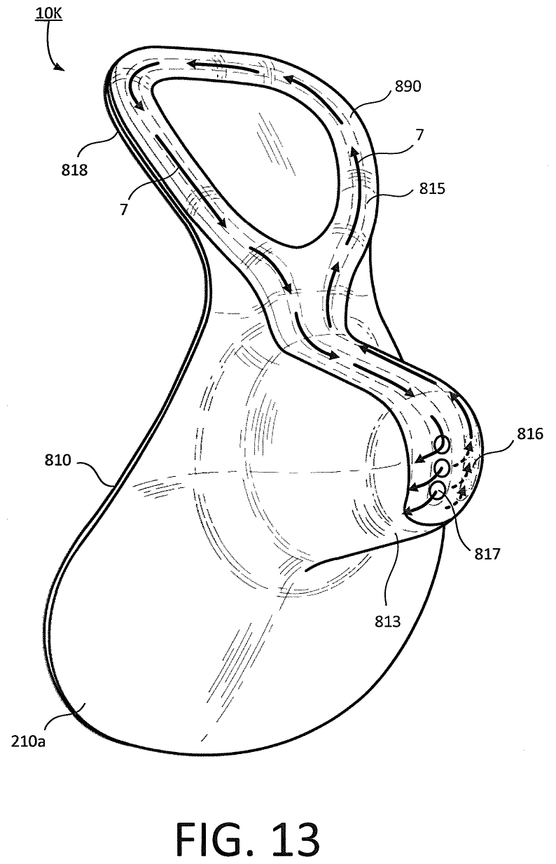

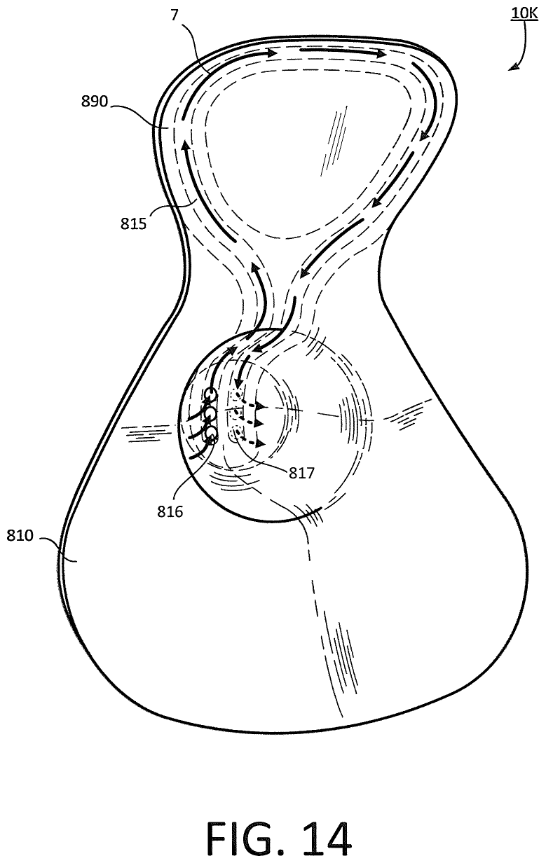

FIGS. 13-19 illustrate another exemplary embodiment for a nipple shield 10K. The form, functionality and similar construction of the nipple shield 10K is similar to the above-described nipple shields. FIGS. 13 and 14 show a front and rear perspective view of the nipple shield 10K with the milk flow path 7 within the nipple shield 10K. FIG.

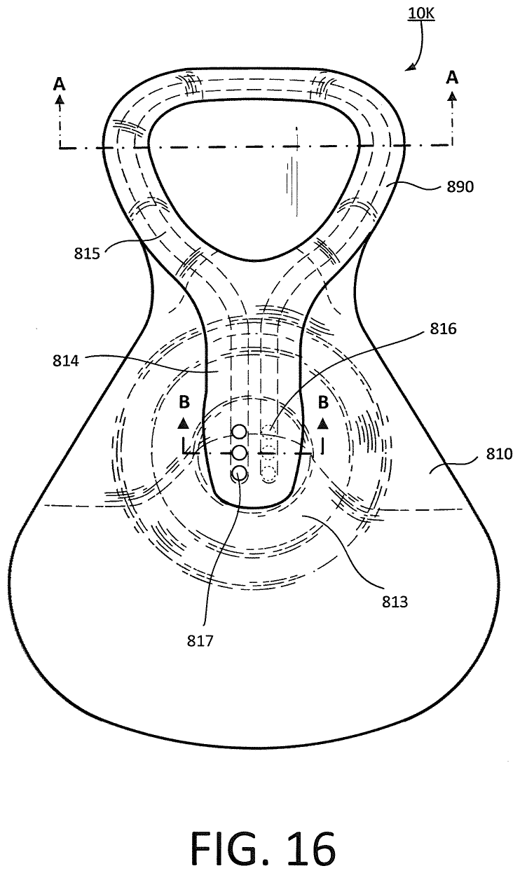

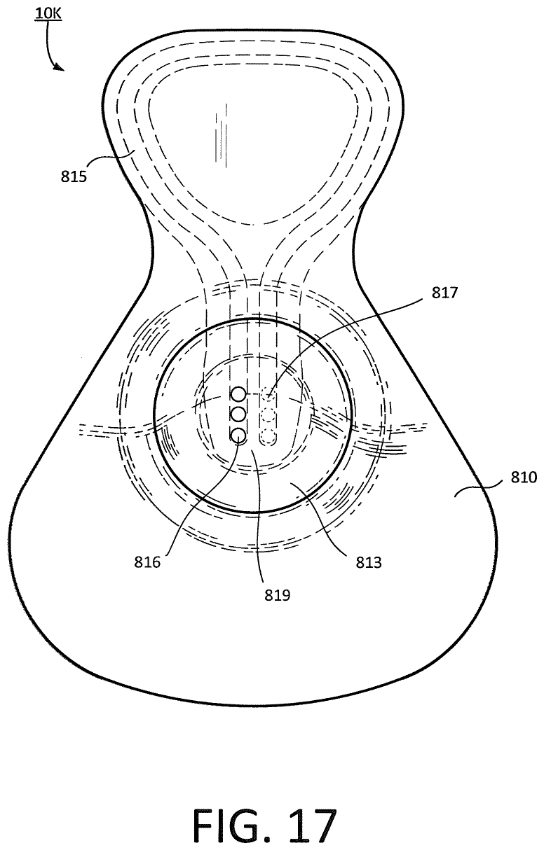

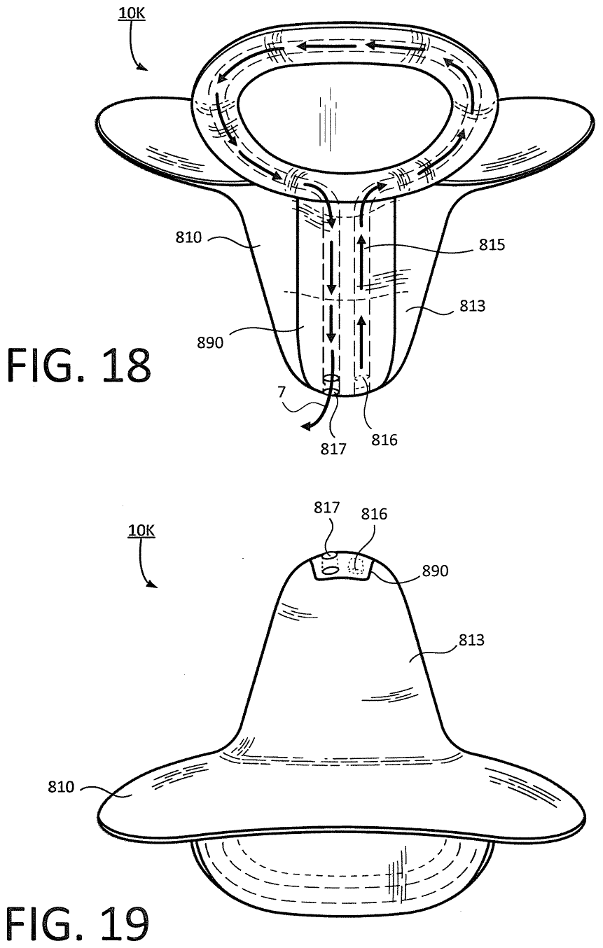

15 is a side X-ray view of the nipple shield 10K depicting the inlet and outlet flow of the milk 7 in the nipple shield 10K. FIGS. 16 and 17 are front and rear views of the nipple shield 10K. FIGS. 18 and 19 show a top and a bottom view of the nipple shield 10K. FIGS. 20-22 and 23-25 illustrate 2 exemplary methods for construction of the nipple shield 10K as will be described in more detail later.

Referring back to FIG. 13, the nipple shield 10K includes a passageway channel cover or channel cover 890 and a base unit 810. The channel cover 890 and the base unit 810 may be formed as a single integrated part or two different parts securely fastened to each other in a permanent manner. The base unit 810 has a wide base with a central protrusion 13 that is adapted to fit over an end of a nursing mothers breast and nipple 12a. The channel cover 890 is constructed of a transparent material, such as silicone, so that the fluid flow of the milk 7 from the mothers breast 12 to the infant 19a can be visibly seen through the transparent material by a mother or caretaker. The channel cover 890 may be a solid or open construction within the circular path made by the passage 815.

In use, the milk 7 flows from the mothers breast through the inlet apertures 816 into and through the passageway 815 within the passageway section 890 and to the outlet apertures 817 to the nursing infants mouth 19. The channels in the passageway 815 extend unobstructed from the inlet apertures 816 that are disposed in an area within an upper end of the dome-like protrusion 813 downward and away from the dome-like protrusion outward in a loop in an outer region 818. The outer region 818 is positioned outside of where the mouth 19 of the infant 19a would cover the dome-like protrusion 813 portion of the nipple shield 10K such as described in more detail in FIG. 1. When the milk 7 flows in the outer region, milk flow 7 can be visibly seen as it travels through the passages 815 in the nipple shield 10K ensuring to the mother or caretaker that the infant is ingesting an amount of milk 7.

Numerous advantages are found by a nursing mother in realizing that her infant is receiving an ample supply of her breast milk. A sense of calm and reassurance by the mother knowing that her infant is ingesting her breastmilk reduces anxiety and/or fear felt by new mothers starting out on their breast-feeding journey. Additional benefits include the creation of a strong emotional connection between the mother and infant. Breastfeeding can also help the mother's body return to its pre-pregnancy state, and may reduce the risk of breast cancer, high blood pressure, diabetes, and cardiovascular disease. A significant advantage can also be realized because breastfeeding is low- cost convenience as it doesn't require preparing bottles or mixing formula. Breastfeeding also helps a mother recover more quickly from her childbirth. Breastfeeding produces oxytocin, which helps the uterus contract and return to its normal size. Another benefit of receiving the breast milk is that it contains antibodies that help the baby's immune system fight off viruses and bacteria and other illnesses and diseases, even long after breastfeeding has stopped.

The shape of the nipple shield is constructed to be fit over the breast 11 of a woman. The shape can be adapted to fit a variety of different sizes and shape breasts, nipples and areolas. The nipple shield 10K may be a thin body 810 constructed from a translucent thin flexible material layer. Preferably, the nipple shield 10K is form fitting and comfortably wearable over the areola and nipple 12a of the nursing mother. The thin transparent flexible material of the nipple shield 10K may be a silicone or any other suitable material which is non-absorbent, non-irritating to the skin and sufficiently flexible to be worn comfortably by the mother on her breast 11.

As shown in FIG. 15, during breastfeeding, an outer surface 814 of the nipple shield 10K faces outward, and comes into contact with, the lips and mouth 19 of the baby 19a. The outlet apertures 817 are disposed on, and in fluid communication with the outer surface 814 of the nipple shield 10K. The nipple shield 10K also depicts an inner surface 819 of the nipple shield 10K facing inward that comes into contact with the inner cavity 14 that collects the milk 7 from the mothers breast 11. The inlet apertures 817 are disposed on the inner surface 819 and are in fluid communication with the inner surface 819 of the nipple shield 10K.

The nipple shield 10K is constructed to include a base unit 810 having a dome-like protrusion 813 disposed in a central region of the base unit 810. In use within the infants mouth 19, the dome-like protrusion 813 defines a cavity 14 between the mothers nipple 12a and an inner surface 819 of the nipple shield 10K.

In use, the cavity 14 area disposed between the mother's nipple 12a and the dome-like protrusion 813 fills with milk 7 from the mother's breast 11. During breastfeeding, the infant sucks on an outer surface 814 of the dome-like protrusion 813 to create a negative pressure in the passageway 815. The negative pressure draws milk 7 to flow into the passageway 815 from the cavity 14. The milk 7 is drawn into the inlet apertures 816 through the passageways 815 and through the outlet aperture 817 and into the nursing infants mouth 19. The negative pressure generated also forces the thin flexible layer 879 of the nipple shield 10K to adhere by suction to the mother's breast 11, thereby sealing the nipple shield 10K preventing milk 7 from escaping externally from the cavity 14 within the dome-like protrusion 813.

FIGS. 16 and 17 show exemplary inlet aperture 816 and outlet aperture 817 hole patterns. As shown in FIGS. 15 and 16, the outlet apertures 817 are disposed on an outer surface 814 of the nipple shield 10K. As shown in FIGS. 15 and 17, the inlet apertures 816 are disposed on an inner surface 819 of the nipple shield 10K. In use, the milk 7 flows from within the cavity 14 under the dome-like protrusion 813 into the inlet aperture (as shown in FIG. 14), through the passageways 815 in the passageway section 890 to outlet apertures 817. The milk flow 7 path continues under the vacuum created by the infant 19a into the infants mouth 19.

Various methods may be provided for attaching the channel cover 890 to the base unit 810 of the nipple shield 10K, such as for example using a glue, a bonding process, a plasma bonding process and/or any other bonding process capable of fastening the channel cover 890 to the base unit 810.

FIGS. 20-22 illustrate one exemplary construction and attachment method for attaching the channel cover 890 to the base unit 810. As shown, the channel cover 890 and the base unit 810 may be made separately and fastened together during assembly. That is, a cover surface 892 of the channel cover 890 would be aligned with and bonded to a base surface 812 of the base unit 810. Both the cover surface 892 and base surface 812 have smooth surfaces adapted to be aligned and lay flush with each other to define the passages 815.

According to this embodiment, the passages 815 are imprinted and recessed into the cover surface 892 within the channel cover 890. As shown in FIG. 20, the base surface 812 extends from a lower portion of the base unit 810 up along a portion of the dome-like protrusion 813 to an upper end. At the upper end of the dome-like protrusion 813, a set of inlet apertures 816 are provided therein to receive milk 7 under suction pressure from within the cavity 14 (shown in FIG. 15).

FIGS. 21 and 22 further illustrate the interconnected structure created when the cover surface 892 of the channel cover 890 is attached to the base surface 812 of the base unit 810 as depicted at sections A-A (FIG. 21) and section B-B (FIG. 22) in FIG. 16. As shown in assembly, the passages 815 for milk flow 7 are constructed as channels disposed between the channel cover 890 and the base unit 810. As shown in FIG. 22, the passages 815 are constructed to communicate the inlet aperture 816 and outlet aperture 817 for milk flow 7.

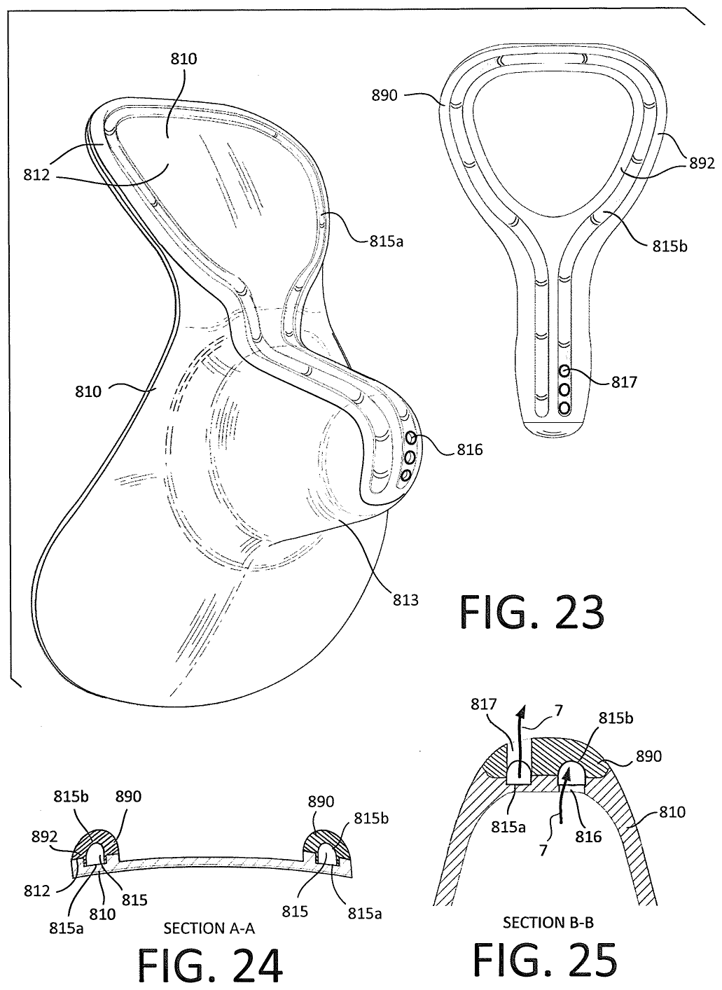

FIGS. 23-25 illustrate another exemplary construction and attachment method for attaching a passageway section 890 to a base portion 810. That is, a cover surface 892 of the channel cover 890 would be aligned with, and bonded to a base surface 812 of the base unit 810. Both the cover surface 892 and base surface 812 have smooth surface portions adapted to be aligned and lay flush with each other to define the passages 815 in the nipple shield 10K.

As shown herein, the passageway section 890 and the base portion 810 may be made separately and fastened together during assembly. According to this embodiment, the passages 815 are imprinted and recessed into both the cover surface 812 within the channel cover 890 and the base surface 812 within the base unit 210. As shown in FIG. 23, the base surface 812 has a smooth surface that extends from a lower portion of the base unit 810 up along a portion of the dome-like protrusion 813 to an upper end. At the upper end of the dome-like protrusion 813, a set of inlet apertures 816 are provided therein to receive milk 7 under suction pressure from within the cavity 14 (shown in FIG. 15).

FIGS. 23-25 illustrate the passage 815 being imprinted and recessed partially within both the cover surface 892 of the channel cover 890 and the base surface 812 of the base portion 810. A first passageway portion 815a of the passage 815 is imprinted or recessed into the base unit 810 within the base surface 812. The first passageway portion 815a extends within the base surface 812 in a return loop along the lower portion of the base unit 810 and then up along a portion of the dome-like protrusion 813 to the upper end.

A second passageway portion 815b is imprinted or recessed into the cover surface 892 within the channel cover 890. The second passageway portion 815b mirrors the shape of the first passageway portion 815a and also extends in a return loop along the channel cover 890. In assembly, the first passageway portion 815a and the second passageway portion 815b mate and overlap with each other to define the complete passageway 815 that connects the inlet apertures 816 to the outlet apertures 817 as shown in FIGS. 24-25. It is to be understood that the passages 815 may be constructed in any portion of the nipple shield 10K.

FIGS. 9, 20 and 23 depict a two-piece nipple shield construction. One exemplary method of manufacture for a nipple shield is by fabricating the channel cover separate from the base unit and then permanently fastening them together as a single integrated unit. Various methods for attaching the channel cover 90, 890 to the base unit 70, 810 are possible, for example but not limited to, an adhesive, liquid silicone rubber, cyanoacrylate adhesive, a plasma bonding process and/or any other suitable method for bonding two pieces of silicone together.

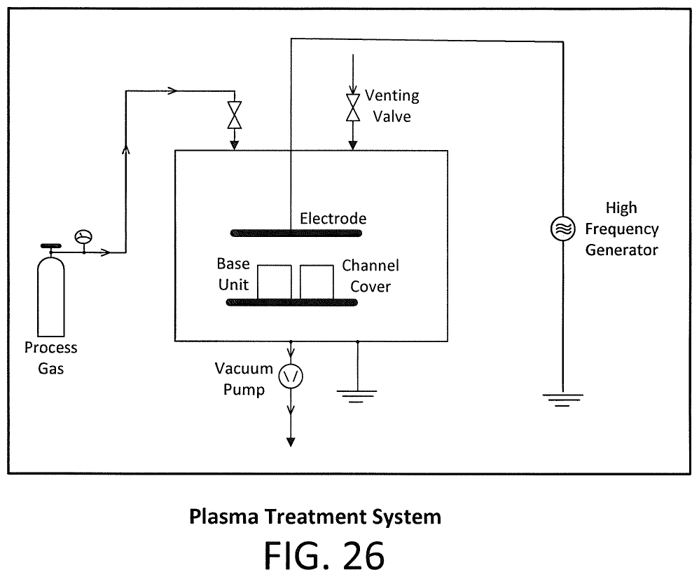

FIGS. 26-32 illustrate an exemplary plasma bonding process that may be used to bond the channel cover 890 to the base unit 810 to form an integrated nipple shield. The base unit 810 and the channel cover 890 may be made from silicone. Between the two pieces of silicone, that is, a base surface 812 and a cover surface 892 may be bonded together under a plasma surface treatment process that modifies the surface properties in such a way as to cause strong adhesion of each of the surfaces 812, 892 to each other.

Plasma bonding involves the use of plasma (an ionized gas, such as oxygen) to treat the surfaces 812, 892 of the silicone nipple shield 800 that are to be bonded. The plasma treatment increases the surface energy by ionizing the surface by ultra-fine cleaning of organic contaminants from the silicone surfaces 812, 892 creating chemical bonding anchors or sites for adhesion. The plasma process causes mechanical and molecular changes to the surfaces 812, 892 of the silicone treated. Through the plasma process, watertight covalent bonds are formed at the attached silicone surfaces 812, 892. The plasma is generated by applying a strong electric field to a gas (like argon, nitrogen, oxygen, etc.), causing it to ionize. This plasma contains various charged particles, including ions, electrons, and radicals, which interact with the silicone surfaces 812, 892 treated.

Particularly, under the plasma process, a continuous application of energy is applied to a first silicone surface (e.g, the base surface 812) of a first piece that is to be bonded to a second silicone surface (e.g, the cover surface 892) of a second piece. The plasma treatment converts a lower surface energy on the surfaces 812, 892 to a higher energy surface by ionizing the surfaces 812, 892 and removing contaminants from the surfaces 812, 892 and attaching oxygen (polar) containing molecules to the surfaces 812, 892. The continuous application of the plasma process causes the temperature on the impinged surfaces 812, 892 to rise and undergo a process from a solid-state to liquid, gas and then plasma. The application of energy causes the existing shell of an atom on the impinged surfaces 812, 892 to break up and become electrically charged. Excited particles and molecule fragments are formed (negatively charged electrons and positively charged ions, radicals) on the impinged surfaces 812, 892 undergoing this process. This mixture is plasma or the fourth state of matter (aggregate state) that derives from a gas.

The initial cleaning process in the plasma bonding process is a critical step to ensuring a strong bond. The cleaning of the base surface 812 and the cover surface 892 at the microscopic level for the presence of contaminants beneficially increases the effectiveness of the bonding between the two surfaces 812, 892 treated for bonding. These contaminants can range from natural oils, grease, wax, organic or inorganic contaminants or other residues from previous processing steps or environmental dust and debris.

During the plasma cleaning process, high-energy ions and electrons in the plasma phase interact vigorously with these contaminants on the surfaces 812, 892, effectively breaking them down or volatilizing them ensuring that the surfaces 812, 892 of the silicone are free of any materials that could negatively impact the adhesion during the plasma bonding process between the two pieces of silicone surfaces 812, 892. The contaminated layer can be irradicated in a number of ways including: by chemically attacking with oxygen or air; by low-pressure and heating on the surface contaminants partially evaporate; by breaking down energy-rich particles in plasma contaminants into smaller molecules that are then sucked off the surfaces 812, 892; and/or using UV radiation to destroy contamination.

Once the surfaces 812, 892 are cleaned, the subsequent bonding steps are conducted on purely clean surfaces, which is integral for the strength and durability of the final bond between the two pieces of silicone surfaces 812, 892. As a result, the tensile and shear strength of the attachment of the silicone cover surface 892 to the silicone base surface 812 is superior to regular adhesives.

Following the cleaning phase, the plasma process facilitates the activation of the silicone surfaces 812, 892 in preparation for the successful bonding between the first and second silicone surfaces 812, 892 by increasing its surface energy. The plasma used in plasma bonding may contain various active species, including radicals, which are highly effective in modifying the surfaces 812, 892 at a molecular level. During plasma activation, these radicals react with the material's surfaces 812, 892, forming new functional groups. These groups are chemically reactive and when the first and second silicone surfaces 812, 892 are located adjacent to each other, strong chemical covalent bonds are formed at a molecular level between the cover surface 892 and the base surface 812.

FIG. 26 shows a basic schematic for a plasma system implemented as a low-pressure plasma system including a vacuum chamber having an electrode and a workpiece (or channel cover 890 and a base unit 810). As shown, the plasma system is adapted include a vacuum chamber, a vacuum pump, receive a process gas, a venting valve and a high frequency generator for plasma creation. Although the configuration for the plasma system can differ based on the desired volume, size and quantity of the nipple shield components to be treated at once.

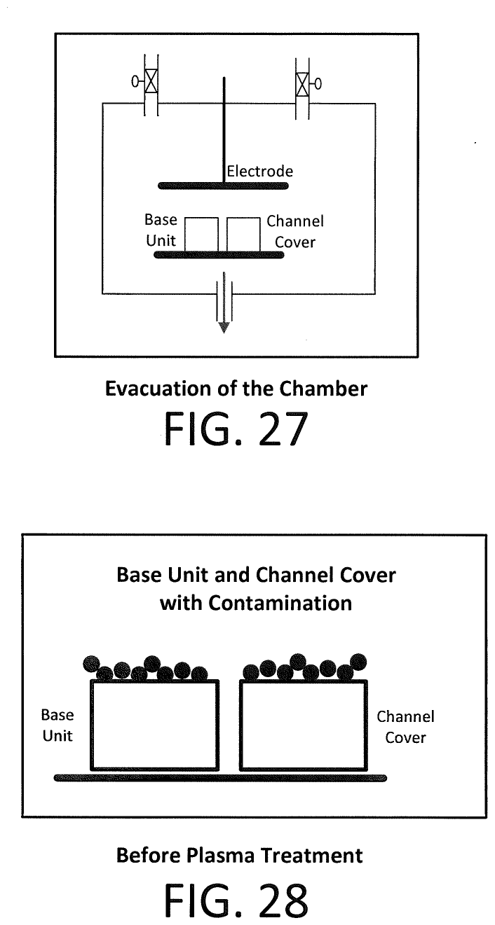

FIGS. 27-32 shows the basic process steps of the plasma process applied to the surfaces 812, 892. In a first step, a channel cover 890 and a base unit 810 having their base surface 812 and the cover surface 892 positioned within the vacuum chamber for direct treatment by the plasma process. As represented in FIG. 28 before the plasma treatment is applied, the surfaces 892, 812 enter the chamber with substantial contamination, such as carbon or the like. In FIG. 27, evacuation of the chamber is performed by creating a low vacuum pressure in the vacuum chamber by a vacuum pump and/or other means. The vacuum pressure may be applied to the vacuum chamber at a predetermined level. For example, the working vacuum pressure in the vacuum chamber may be in the field of 0.1 to 1.0 mbar.

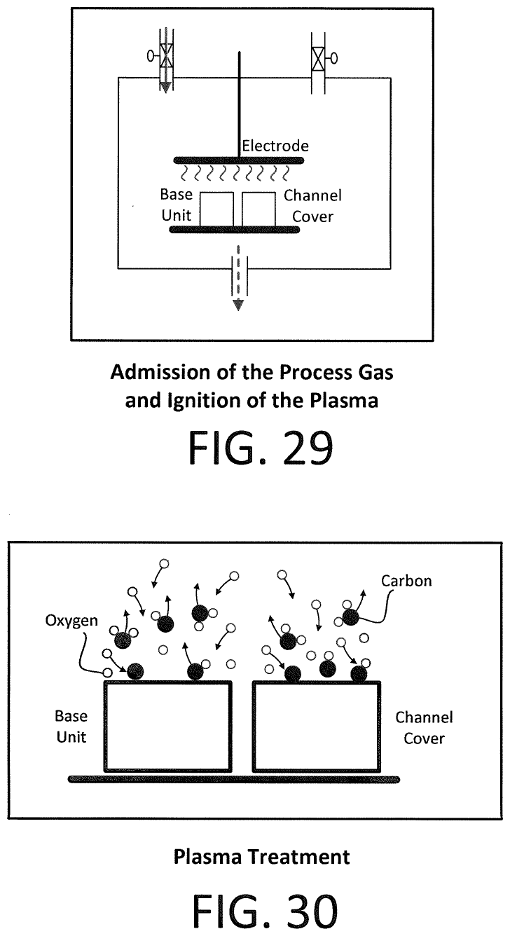

In a second step shown in FIGS. 29-30, a process gas (e.g. oxygen) is fed into the vacuum chamber. When the working pressure is achieved, the high frequency generator is switched on and the process gas in the chamber is ionized. That is, the atoms or molecules in the gas are not neutral, but instead carry an electrical charge when the gas gains or loses electrons in conjunction with other chemical changes. As shown in FIG. 30, the cover surface 892 and the base surface 812 to be treated and/or cleaned are exposed to and impinged by the ionized process gas under the plasma process. The plasma system will receive continuously fresh process gas to the surfaces 812, 892 while the contaminated gas is simultaneously sucked off and removed out of the vacuum chamber thereby cleaning the surfaces 892, 812 to be bonded together. The plasma treatment process is a surface cleaning coating process using the process gases. That is, the oxygen gas which oxidizes the surfaces 812, 892 creates silicone hydroxide bonds under the plasma cleaning process. The plasma treatment introduces polar, reactive functional groups to the silicone surfaces 812, 892, enabling water-tight covalent bonding.

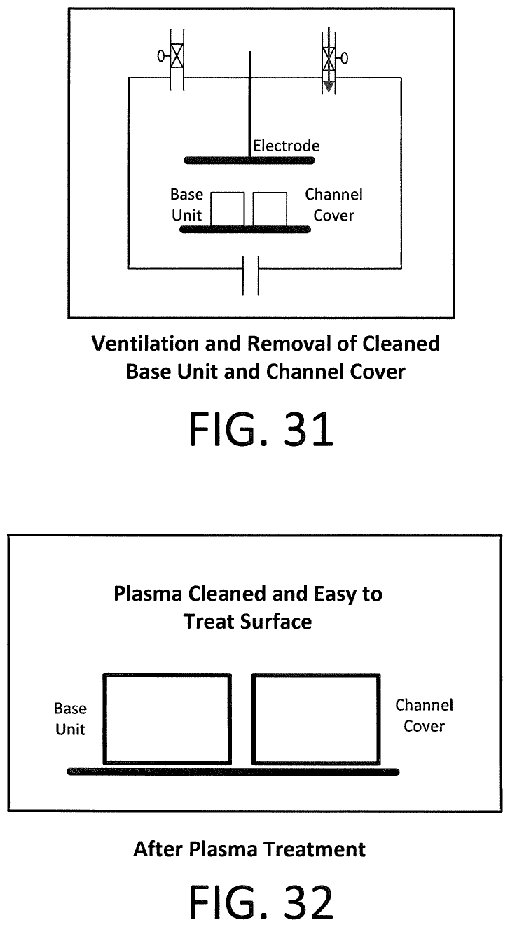

In a third step shown in FIGS. 31-32, the vacuum chamber is ventilated and the channel cover 890 and the base unit 810 are removed from the vacuum chamber. Fresh out of the vacuum chamber, the cover surface 892 of the channel cover 890, and the base surface 812 of the base unit 810 are cleaned free of contaminants and have an increased surface energy that was generated by the plasma treatment. The high surface energy of the cover surface 892 and the base surface 812 over time will eventually settle and reconfigure to a lower energy state establishing the permanent covalent bond between the surfaces 892, 812. Alternatively, an increased temperature (such as 80-100 degrees Celsius) can be applied to accelerate the activation of the covalent bonding process between the two surfaces 892, 812 for a predetermined period of time (e.g., 60 seconds) during the settling time. This can also be done using a hot plate, an oven and/or other heating mechanism according to this subject disclosure.

Referring back to FIGS. 20 and 23 depict the channel covers 890 and the base units 810 just removed from the vacuum chamber having highly charged surface energies on their respective cover surface 892 and base surface 810 ready for covalent bonding interaction to each other. The cover surface 892 of the channel cover 890 is then aligned with the base surface 812 of the base unit 810 and pressed tight to each other so that covalent bonding between the two high energy surfaces 892, 812 can occur securely attaching them to each other to define the passageways 815 as shown in FIGS. 21-22 and FIGS. 24-25. The two highly excited high energy surfaces 892, 812 interact together forming strong covalent bonds between the surfaces 892, 812 to create a sufficiently strong permanent bond between the cover surface 892 and the base surface 812. That is, as the two high energy surfaces 892, 812 are places against each other, various covalent bonds are formed by chemical reactions that occur between the two high energy surfaces 892, 812 in which electrons are shared to form electron pairs between atoms known as shared pairs or bonding pairs. Eventually, the sharing of electrons allows each atom to attain the equivalent of a full valence shell, corresponding to a stable electronic configuration and providing a permanent secure bond between the surfaces 892, 812, thereby producing an integrated nipple shield 800.

An advantage of this plasma bonding method attachment between the surfaces 892, 812 is a perfect bonding of the mating flat surfaces between the cover surface 892 and the base surface 810 without the use of an adhesive. The elimination of an additional adhesive is beneficial in that it will effectively prevent the potential for spill over into the channels in the passages 815 thereby constricting the passages and/or potentially contaminating the milk flow during use.

Various options and parameters relating to the method of plasma creation can be alternatively utilized. The energy, necessary for plasma creation, can be coupled to the chamber in different ways. An electrode may be used to generate plasma. The electrode may be placed within the plasma chamber. Alternatively, a microwave machine may be built into a magnetron. The magnetron may be an electron-tube-oscillator that oscillates at a fixed frequency that directs microwaves onto a glass or ceramic window that enters the vacuum chamber.

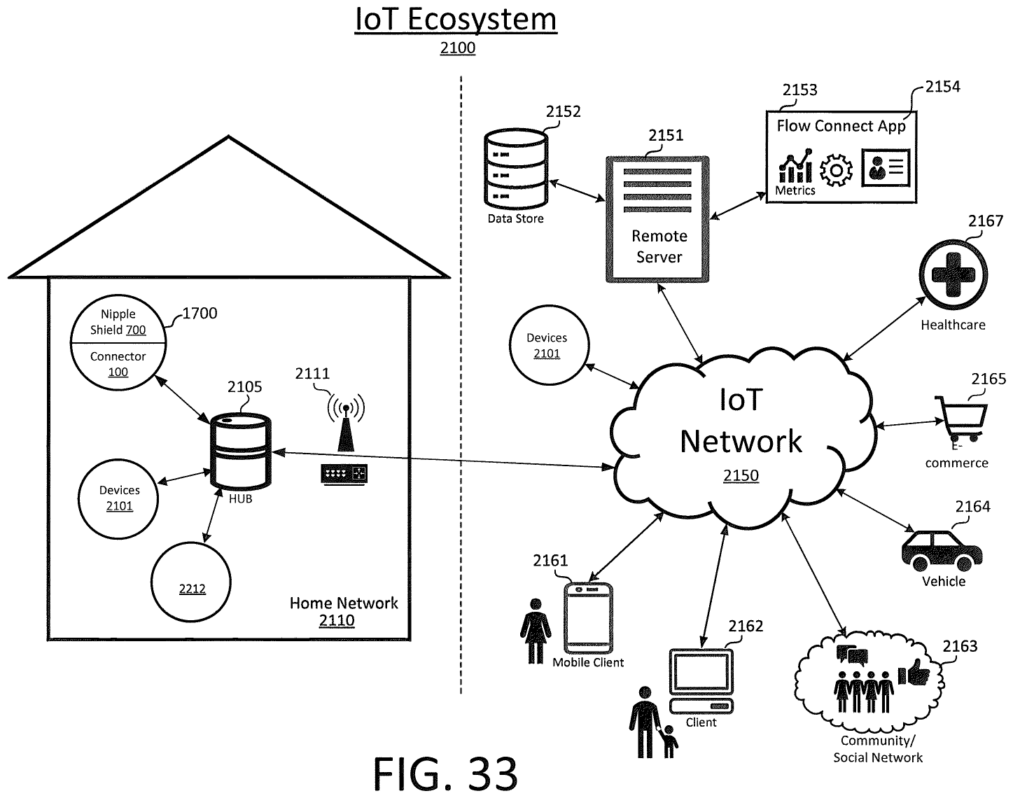

Referring back to FIGS. 10-12 and to FIGS. 33-35, the various flow measurement connectors 100 may be integrated with a remote computer, and/or embodied as an application in a remote server and/or as a mobile application, adapted to transmit signaling to and from a multi-task nipple detection unit. FIG. 33 illustrates an exemplary ecosystem for use with this subject disclosure. A multi-task nipple detection unit 1700 may include a multi-task nipple shield 700 and a connector 100. The multi-task nipple detection unit 1700 may be provided that is based on the measurements and/or parameters detected by the various connectors 100 described above and implemented with the multi-task nipple shield 700.

FIG. 33 illustrates an exemplary internet of things (IoT) ecosystem 2100 or network of objects (e.g., the multi-task nipple detection unit 1700) that are capable of connecting and exchanging data over the internet or other similar network infrastructure. The multi-task nipple detection unit 1700 includes a nipple shield 700 and a connector 100. The IoT ecosystem 2100 may include one or more multi-task nipple detection unit 1700 or other devices 2101 in a home network 2110 or other location.

The multi-task nipple detection unit 1700 or other devices 2101 may be any of the above listed connectors 100 or the like incorporated with the nipple shield 700 as described above or contemplated in the future with other suitable devices according to this subject disclosure. The multi-task nipple detection unit 1700 may be connected to an IoT hub 2105. The hub 2105 may be adapted to control the various multi-task nipple detection units 1700 included in the IoT ecosystem 2100. The hub 2105 may be integrated into a smart device, or the hub 2105 may be a standalone device. Alternatively, a home router 2111 may serve as a hub into which all of the multi-task nipple detection units 1700 in the home network 2110 are connected and communicate to and from into the IoT network 2150. The hub 2105 may be adapted to connect to the IoT network 2150 through a wired or wireless internet connection. The hub 2105 may have a processor, memory, a communication module and a locational unit having a global navigation satellite system (GNSS) receiver. The GNSS receiver may be a global positioning system (GPS) receiver. The processor may be electrically coupled to the memory, the communication module and the other electrical components attached therein by one or more high-speed buses.

The home network 2110 may be a bespoke network or a network adapted to run on common IoT platforms such as Google Cloud, IRI Voracity, Particle, Salesforce IoT Cloud, IBM Watson IoT, ThingWorx, Amazon AWS IoT Core, Microsoft Azure IoT Suite and the like. The IoT network 2150 may be a distributed network and may be partially or entirely substantiated on one or more remote servers 2151.

The remote server 2151 may have a processor, memory and a server communication module coupled together by one or more high speed buses. The remote server 2151 may be connected to one or more database 2152. The database 2152 may be stored in the memory of the remote server, or may exist on a separate server, a virtual server or hosted in a cloud network. The remote server memory 2153 may run an application 2154 (e.g., herein Flow Connect App) adapted to communicate with the multi-task nipple detection units 1700 or other devices 2101 connected within the home network 2100 or connected to the IoT network 2150.

The application 2154 is capable of sending and receiving communications to and/or from the multi-task nipple detection units 1700 or other devices 2101. The application 2154 includes various associated routines, algorithms, machine learning or artificial intelligence (AI) applications, data, software, logs, or a combination thereof. The application 2154 is a specific smart device program or stand alone software that is specifically designed to interact with the subject disclosure as described herein.

The IoT network 2150 may be connected to one or more client devices and one or more service providers. The client device may be a portable computing device 2161 such as a smartphone, a desktop, a laptop, a tablet, a smartwatch, an entertainment device, and/or a voice-enabled smart device such as a Google home device, Amazon Echo device, Apple smart device, Facebook smart device, Samsung smart device, LG smart device, Sonos smart device, or another smart home controller 2162 or hub device. The IoT network 2150 may be connected to one or more vehicles 2164 and/or one more in-vehicle computers. The IoT network 2150 may be connected to one or more service providers. The connected service provider may be a healthcare provider 2167, such as a doctor, a doctor's office, a hospital, or other health provider. The service provider may be one/or more emergency services such as the police or fire department. One or more community/social networks 2163 may be connected to the IoT network 2150. The social network 2163 may be a network or community of parents, and other caregivers that share stories and child rearing advice. The community may share preferred settings for the various devices 2101, 2161, 2162, etc. based on the best practice learned with their children and the like. Lactation consultants or other childcare experts may be included in the social network and the network may feature informative blogs, inquiry capabilities, recommendations, and articles for assisting caregivers. The IoT network 2150 may be connected to one or more Ecommerce platforms 2165, such as Munchkin, Amazon, Walmart and the like for facilitating the purchase of essential and/or desired products for assisting with childcare duties.

The IoT network 2150 may include any multi-hop network or wide area network (WAN) that covers cities, states, regions, countries or other locations. The IoT network 2150 may include any number of wireless local area networks (WLANS), including those established under IEEE's 802.11 protocol or its successors. The WLANS may include a number of wireless-fidelity (WiFI) networks. The IoT network 2150 may incorporate cellular networks such as 2G, EDGE, 3G, H, H+, 4G, 5G, long-term evolution (LTE) networks, the internet, a satellite network, a Starlink network and/or any other suitable network capable of signaling within the IoT Ecosystem 2100.

The home network 2110 within the IoT ecosystem 2100 may be adapted to work with other devices, various sensors and/or other smart home controllers, voice-assisted devices 2212 (GOOGLE, ECHO, ALEXA, etc.) or other hub devices now known or later discovered. The IoT hub 2105 may be adapted for use in the nursery and/or use in another room in the house, such as the kitchen, living room, bathroom or a caregiver's bedroom. The home network 2110 may include other smart devices located in the house or environment.

The multi-task nipple detection units 1700 or other devices 2101 in the home network 2110 may be in wireless communication directly with the hub, connected client devices, sensors, and/or the connected devices through a short-range wireless communication protocol, such as a Bluetooth protocol, Bluetooth Low Energy (BLE) protocol, ZigBee protocol, near-field communication (NFC) protocol, any combination thereof, and/or any of the communication protocols previously discussed above.

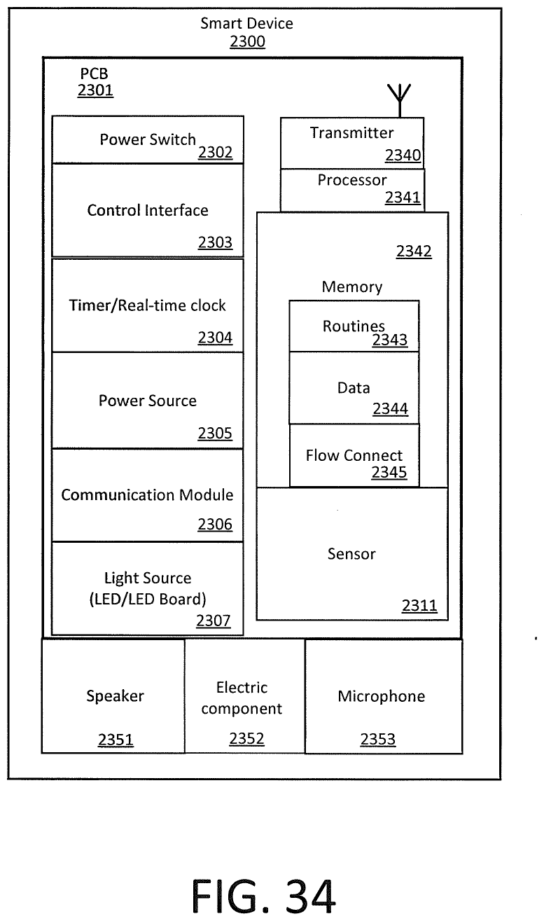

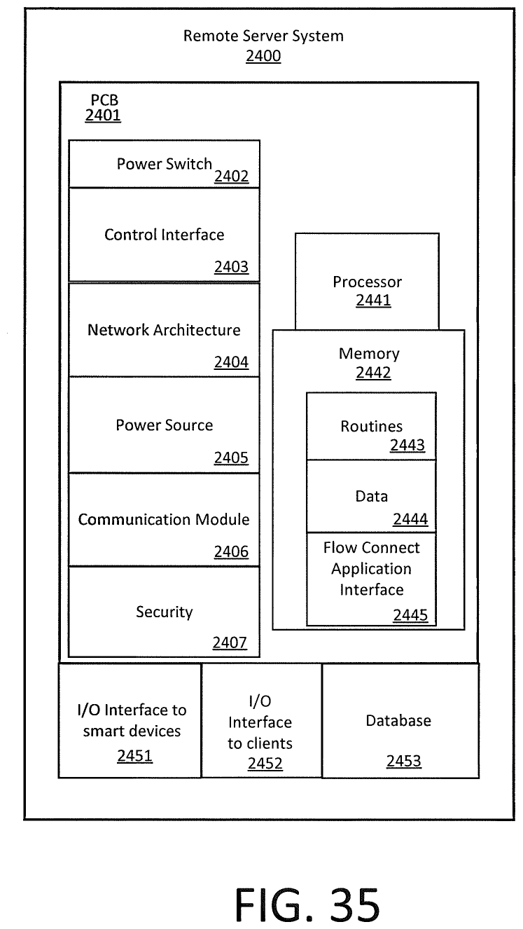

FIG. 34 details a block diagram of a common smart device 2300 printed circuit board (PCB) 2301 comprising a number of chips, modules, integrated circuits (ICs), sensors, interfaces and high-speed buses. For example, the PCB may include a power switch 2302, a user control interface 2303, a timer and real-time clock IC 2304, power source 2305, a communication module 2306, one or more sensors 2311, a processor 2341 and memory 2342. Other components 2352 may be electrically coupled to the PCB 2301 and incorporated into a device circuit. For example, a light source, a speaker 2351, a vibrating device, a microphone 2353, a digital clock, ambient lights, a capacitive touchscreen and/or other components may be incorporated into the device circuit. The light source may be one or more LEDs electrically coupled to an LED circuit board. The LED circuit board may be the PCB 2301 or a separate LED circuit board that is electrically coupled to the PCB. The LED board may be separated from the PCB 2301 by a gap or distance and may be positioned above or elevated from the PCB. The PCB 2301, the LED Board and the other integrated electrical components may be coupled to one another by clips, clasps, adhesives, fasteners, screws, thread connections, interference fit, heat staking, thermoplastic staking by laser welding or ultrasonic welding, or a combination thereof. The PCB 2301 or LED board may be attached to the device housing through conventional means such as studs, posts, ribs, bosses that extend from the housing and attach to the boards through holes, slots, staking, interference fit, or a combination thereof.

The device processor 2341 may execute software or routines 2343 stored in the memory 2342 to execute the methods described herein. The processor 2341 may be an embedded processor, a microprocessor, a logic circuit, a hardware finite-state machine (FSM), a digital signal processor (DSP), or a combination thereof. The processor 2341 may include one or more central processing units (CPUs), graphical processing units (GPUs), application-specific integrated circuits (ASICs), field-programmable gate arrays (FPGAs), or combination thereof.

The device memory 2342 may store software, AI or machine learning algorithms, routines, microcode, firmware and logs. The memory 2342 may be internal memory or external memory such as an external storage unit. The memory 2342 may be non-volatile memory such as read-only memory (ROM), NVRAM, Flash memory and/or disc storage. The memory may be volatile memory such as random-access memory (RAM), DRAM and/or SRAM. Data 2344 and the MunchkinIQ application 2345 may be included in the device memory 2342. The memory 2342 may be adapted to receive software and firmware updates from the IoT network.

The device communication module may include one or more wired or wireless communication interfaces. The communication module may be a network interface card of the smart device. The communication module may be a wired or wireless modem and may support WiFI, 3G, 4G, LTE, Bluetooth and/or radio technologies. The communication module may include a transceiver 2340 having a receiver and an antenna capable of sending and receiving signaling within the IoT ecosystem 2100. The communication module may communicate with a wide area network (WAN) in order to transmit or receive packets, data, messages and/or instructions.

FIG. 35 is a block diagram drawing of a remote server system 2400 embedded with the application 2154. The server may have a processor 2441, memory 2442, a server communication module 2406, and/or other electrical components forming a circuit and in communication through high-speed buses.

The server processor 2441 may execute software or routines 2443 stored in the server memory 2442 to execute the methods described herein. The processor 2441 may be an embedded processor, a microprocessor, a logic circuit, a hardware finite-state machine (FSM), a digital signal processor (DSP), or a combination thereof. The processor 2441 may include one or more central processing units (CPUs), graphical processing units (GPUs), application-specific integrated circuits (ASICs), field-programmable gate arrays (FPGAs), or combination thereof.

The server memory 2442 may store software, routines 2443, logs, network architecture, communication protocols, encryption protocols, security protocols for secure communication through the IoT network and between the remote servers and endpoint devices, data 2444 for data processing protocols and instructions and other features and software associated with the application 2445 and network. The memory 2442 may be internal memory or external memory such as an external storage unit. The memory 2442 may be non-volatile memory such as read-only memory (ROM), NVRAM, Flash memory and/or disc storage. The memory 2442 may be volatile memory such as random-access memory (RAM), DRAM and/or SRAM. The memory 2442 may be adapted to send software and firmware updates to the various devices connected through the IoT network. The server memory 2442 may include or be connected to one or more databases for the storage and processing of data 2444 associated with the application 2445.

Further components on the PCB 2401 may include, but are not limited to, power switch 2402, control interface 2403, network architecture 2404, power source 2405, communication module 2406, security 2407, I/O interface to smart devices 2451, I/O interface to clients 242, and database 2453.

The server communication module 2406 may include one or more wired or wireless communication interfaces. The communication module may be a network interface card of the server. The communication module 2406 may be a wired or wireless modem and may support WiFI, 3G, 4G, LTE, Bluetooth, BLE and/or radio technologies. The communication module 2406 may include a receiver and an antenna. The communication module 2406 may communicate with a WAN, WLAN or other network in order to transmit or receive packets, data, messages and/or instructions through the server communication module.

The application 2445 may be adapted to execute one or more graphical user interface (GUI) on the client devices or other smart devices connected to the IoT network. The app may be stored in the memory 2442 of the remote serve, or the memory of the client devices or smart devices. The application software may also be hosted in a cloud server. The application GUIs may allow a user to interact with, and control, the application and related software.

The application may be also be adapted to collect data related to the IoT ecosystem, connected devices, the multi-task nipple detection units and various users of the application. The application may be adapted to generate suggestions, advice and education information for caregivers using the data collected, and/or other sources. The application may be adapted to identify a caregiver's problem or unique situation and generate advice or education related to sleep training, nutrition, helpful childcare products, growth and development milestones, and the like. The application may send push notifications to client devices and smart devices to provide advice, alerts and other notifications. The application may be linked to one or more ecommerce platforms in order to facilitate the purchase of necessary and desired products. The application may incorporate regional information such as weather and air quality index (AQI) and send notifications related to this information to the users. The application may include a user community that pools caregiver experiences, stories, advice, and other information related to their use with the IoT ecosystem and childcare. For example, the user community may share personalized set-up configurations and best uses of the connected devices. The application may include a neighbor function for connecting with other caregivers in the area. The application may include various modes, for example, a sleep mode, a nap mode, a deep sleep mode, etc. The application may be adapted to detect, survey and share sales and marketing information with the user based on detected or learned information collected from analyzed data collected. The application may be adapted for use with multiple operating systems including Windows, iOS and Android.

The application may be integrated with artificial intelligence (AI) applications are software programs that use Al techniques to perform tasks. These tasks can range from simple, repetitive tasks to complex, cognitive tasks that require human-like intelligence. The application can use the AI applications to train by processing large amounts of data and understanding the relationship between each specific element. The data can be a data lake in a centralized repository that can store structured and unstructured data at any scale. The data can be stored as-is, without having to first structure the data, and run different types of analytics, from dashboards and visualizations to big data processing, real-time analytics, and machine learning to guide better decisions.

The application may include security features to protect the user's privacy. The application may be adapted to control access to the home network and IoT ecosystem, the IoT network and the connected devices to specific family, friends, neighbors and babysitters. The application may be adapted for use with authentication, encryption and security software that may restrict access to the application and network based upon authorization, user identity, time restrictions, events, and the various modes of the application. The application may include a lock feature that locks the connected devices from use during certain modes or based upon user preference. In one embodiment, a lock feature may be provided to prevent a child from disabling the routine set by the caregiver.

The application may be adapted to turn on, shut off, power off or deactivate the connected smart devices. The application may be adapted to provide GUIs that facilitate and actuate the user's ability to control the various features of the connected devices. The application may be adapted to record and update the GUI and application based upon user actions and inputs on the physical devices. The application may render a firmware update GUI to inform the user that the firmware of a certain connected device is being updated. Updates to the application software or device firmware may be pushed to the client device or smart devices to improve the functions of the application or device and/or introduce new software or device features.

The IoT Ecosystem 2100, as described in detail above has a number of factors and influences. These include, but are not limited to, the application which collects data, work, vehicle, Mobile App (phone, computer tablet, remote control), health care, family networks (sitters, nannies), AI algorithms, security (data share, upload, download), community (blog/best mode, setup, present, neighbor function), and home (nursery, parent's room).

The IoT Display and Other Product Tie-In's (BT/WiFi/Wireless/Wired) may include, but not limited to: Apple TV/Firestick, Google/Echo/Alexa, Emergency 911, Alarm-Phone, Emergency Contact List, and Bose.

The application functions to, but is not limited to: Gather data ID the problem, Give Advice, Education (Sleep, Nutrition, Products, Milkmakers, Partnership with other companies/products, Current and Future Products, Baby Development/Milestones), Push Notifications, E-commerce, Regional Environment Knowledge, Create Community and provide Recommendations-like Ring App (Blog/Best Mode/Setup, Preset mode, Upload/Download Neighbor Functions/Recommendations), Smart Tracking/Recommendations, Sales/Marketing.

Privacy considerations include but are not limited to: Controlled Access (To family/friends/neighbor/sitter), Visibility in Nursery/Camera, Smart Block—Time restrictions/Nursing time, Timed Alert, Authorization Code for access, and Safe List.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. Furthermore, it is appreciated by persons skilled in the art that the present invention is not limited by what has been particularly shown and described hereinabove. Rather the scope of the present invention includes both combinations and sub-combinations of various features described hereinabove as well as variations and modifications thereto which would occur to a person of skill in the art upon reading the above description and which are not in the prior art.

The illustrations and examples provided herein are for explanatory purposes and are not intended to limit the scope of the appended claims. The examples and illustrations described herein clearly define the technical problem being addressed, and highlights concrete technological solutions, emphasizes specific technical details of the implementation of the nipple fluid flow detection systems and methods, and demonstrates practical applications beyond simply processing data. It will be recognized by those skilled in the art that changes or modifications may be made to the above-described embodiment without departing from the broad inventive concepts of the invention. It is understood therefore that the invention is not limited to the particular embodiment which is described, but is intended to cover all modifications and changes within the scope and spirit of the invention.

What is claimed:

1. A flexible device adapted to provide a visual indication of a milk flow from a breast to an infant during nursing, the flexible device comprising:
a base unit having an upper surface and a lower surface, the base unit having a protrusion disposed in a central region adapted to be positioned over a nipple of the breast, wherein the lower surface of the base unit is adapted to lie securely against the breast; and
a channel cover having an upper cover surface and a lower cover surface, the lower cover surface adapted to be attached to the upper surface of the base unit to define a channel passageway that fluidly connects at least one inlet opening in the lower surface of the base unit, and at least one outlet opening in the upper cover surface of the channel cover, and
wherein the channel passageway extends from the protrusion to a remote location so that the milk flow in the channel passageway can be visually observed when a mouth of the infant covers a portion of the flexible device.

2. The flexible device recited in claim 1, wherein the at least one inlet and at least one outlet is disposed adjacent to an apex in the protrusion.

3. The flexible device recited in claim 1, wherein portions of the lower cover surface of the channel cover are attached to the upper surface of the base unit by a plasma treatment process to define the channel passageway.

4. The flexible device recited in claim 1, wherein the plasma treatment process chemically energizes and removes contaminants from the lower cover surface of the channel cover and the upper surface of the base unit such that when the lower cover surface of the channel cover and the upper surface of the base unit are aligned and come into contact a permanent covalent bonding between the surfaces permanently fixes the lower cover surface of the channel cover to the upper surface of the base unit.

5. The flexible device recited in claim 1, wherein the channel cover is permanently attached to the base unit by a plasma treatment process to define a permanently integrated nipple shield.

6. The flexible device recited in claim 1, wherein the flexible device is attached to an electronic connector to detect a characteristic in the milk flow.

7. The flexible device recited in claim 6, wherein the electronic connector evaluates the detected characteristic and returns an output.

8. The flexible device recited in claim 6, wherein the electronic connector communicates the detected characteristic to a remote processor to evaluate the detected characteristic and to return an output.

9. A nipple shield adapted to provide a visual indication of a milk flow from a breast to an infant during nursing, the nipple shield comprising:
a wide base having a protrusion disposed in a central region that is adapted to be positioned over a nipple of the breast, wherein the wide base is adapted to lie securely against the breast; and
a single channel passageway that fluidly connects an inlet opening in a lower surface of the protrusion to an outlet opening on an upper surface of the protrusion, wherein the single channel passageway extends from the inlet opening to a remote location so that all of the milk flow from the inlet opening in the single channel passageway can be visually observed when a mouth of the infant covers a portion of the protrusion.

10. The nipple shield recited in claim 9, wherein more than one inlet opening or outlet opening is provided in the protrusion.

11. The nipple shield recited in claim 9, wherein the inlet opening and the outlet opening are disposed adjacent to an apex in the protrusion.

12. The nipple shield recited in claim 9, wherein the single channel passageway is at least partially translucent such that the milk flow can be visually observed.

13. The nipple shield recited in claim 9, wherein the single channel passageway is fluidly connected to various connectors to detect various parameters in the milk flow, the various connectors are attached at a predetermined location adjacent to a peripheral edge of the nipple shield.

14. The nipple shield recited in claim 9, wherein the single channel passageway is fluidly connected to various connectors to detect or provide indication of various parameters in the milk flow.

15. The nipple shield recited in claim 9, wherein nipple shield is a silicone material that is transparent.

16. The nipple shield recited in claim 9, wherein the wide base and the single channel passageway are constructed as separate parts, and wherein a plasma treatment process:
chemically removes contaminants from an upper surface of the wide base and a lower surface of the single channel passageway; and
highly energizes the upper surface of the wide base and a lower surface of the single channel passageway;
wherein when the lower surface of the single channel passageway is aligned with and placed adjacent to the upper surface of the wide base, the two surfaces chemically interact together forming strong permanent covalent bonds between the surfaces.

17. A nipple shield adapted to provide a visual indication of a milk flow from a breast to an infant during nursing, the nipple shield comprising:
a base unit having an upper surface and a lower surface, the base unit having a protrusion disposed in a central region adapted to be positioned over a nipple of the breast, wherein the lower surface of the base unit is adapted to lie securely against the breast; and
a channel cover having an upper cover surface and a lower cover surface, the lower cover surface adapted to be permanently attached to the upper surface of the base unit by a plasma treatment process to define a channel passageway that fluidly connects at least one inlet opening in the lower surface of the base unit, and at least one outlet opening in the upper cover surface of the channel cover, and
wherein the channel passageway extends from the inlet opening in the protrusion and outward to a remote location and then back to the outlet opening in the protrusion such that the milk flow in the channel passageway can be visually observed in the remote location when a mouth of the infant covers a portion of the flexible device.

18. The nipple shield recited in claim 17, wherein the nipple shield is a silicone material that is transparent.

19. The nipple shield recited in claim 17, wherein the at least one inlet opening and the at least one outlet opening are disposed adjacent to an apex in the protrusion.

20. The nipple shield recited in claim 17, wherein the base unit and the channel cover are constructed as separate parts, and wherein the plasma treatment process is applied to:
chemically remove contaminants from an upper surface of the base unit and a lower cover surface of the channel cover; and
highly energize the upper surface of the base unit and a lower cover surface of the channel cover;
wherein when the lower surface of the channel cover is aligned with and placed adjacent to the upper surface of the base unit, the two surfaces chemically interact together forming strong permanent covalent bonds between the surfaces.
